(19) United States
(12) Patent Application Publication
(10) Pub. No.: US 2010/0292663 A1
(43) Pub. Date: Nov. 18, 2010

(54) Disposable Absorbent Article

(76) Inventors: Gary Dean LaVon, Liberty Township, OH (US); Luke Nogales, Cincinnati, OH (US); Ernesto G. Bianchi, Mason, OH (US); Lisa M. Frederick, Fairfield, OH (US); John Brian Strube, Okeana, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/779,434

(22) Filed: May 13, 2010

Related U.S. Application Data
(60) Provisional application No. 61/178,524, filed on May 15, 2009.

(51) Int. Cl. A61F 13/15

(52) U.S. Cl. 604/367; 604/378; 604/386; 604/385.26

(57) ABSTRACT

A disposable absorbent article may comprise a web that may be folded over at a first fold line forming a first hem, folded over at a second fold line forming a second hem, and folded over at a third fold line and attached in the front and back waist regions to form a first side flap. The web may be folded over at a fourth fold line and attached in the front and back waist regions to form a second side flap. The absorbent article may also comprise a second elastic gathering member attached at or adjacent the side edge of the absorbent article. Also, the absorbent article may comprise laterally opposing deployable belt ears attached to the web in at least one of the waist regions. Each belt ear may be disposed laterally inward until being deployed laterally outward so as to project laterally outward beyond the respective distal edges of the side flaps.

### CROSS REFERENCE TO RELATED APPLICATIONS

This application claims the benefit of U.S. Provisional Application No. 61/178,524, filed May 15, 2009, the substance of which is incorporated herein by reference.

### FIELD OF THE INVENTION

This invention relates to disposable absorbent articles such as disposable diapers and other articles intended for use on incontinent persons.

### BACKGROUND OF THE INVENTION

Disposable absorbent articles are designed to absorb and contain bodily waste in order to prevent soiling of the body and clothing of the wearer, as well as bedding or other objects with which the wearer comes into contact.

As the usage of disposable absorbent articles has expanded, their complexity has increased with the incorporation of additional features serving to enhance their performance and appearance. The costs of the materials and the costs of the manufacturing processes have also increased in conjunction with the increase in complexity. As a result, the prices at which these articles are sold have risen to levels that many potential purchasers around the world cannot afford to pay. Thus, a need exists for a simple disposable absorbent article.

In order to deliver a low cost solution, the absorbent article must be material efficient and the process to make it must be as low cost as possible. In order to deliver a low cost solution, the articles of the present invention utilize a web comprising a continuous layer. The web is then manipulated by folding to deliver a “one piece” integrated chassis. This construction is significantly cheaper and simpler than introducing multiple webs of material and then combining those webs together by adhesives and other means. The single continuous web utilized in the absorbent articles of the present invention described herein form the chassis comprising hems for the inner elastics, the hems for the outer elastics, the side flaps (each comprising the cuff flap and side barrier) and the backsheet in one unitary structure. The low cost solution leverages not only simplicity of design and material efficiency but includes activation to deliver customizeable fit. The activation of the chassis web renders portions of a simple low cost plastic film extensible and somewhat elastic. This extensibility allows for increased flexibility with regard to fit and sizing, as well as delivers significant wearer comfort.

### SUMMARY OF THE INVENTION

A disposable absorbent article may comprise a front waist region, a back waist region, a crotch region between the waist regions, a front waist end edge, and a back waist end edge. Further, a longitudinal axis may extend from a midpoint of the front waist end edge through the crotch region to a midpoint of the longitudinally opposed back waist end edge. The absorbent article may also comprise a web comprising an interior surface and an exterior surface. The article may further comprise an absorbent assembly comprising an interior surface and an exterior surface

The web may be folded over at a first fold line forming a first hem, folded over at a second fold line forming a second hem, and folded over at a third fold line and attached in the front and back waist regions to form a first side flap. The third fold line may form a first side edge of the absorbent article. The web may be folded over at a fourth fold line and attached in the front and back waist regions to form a second side flap. The fourth fold line may form a second side edge of the absorbent article.

The first side flap may comprise the first hem and the second side flap may comprise the second hem. Each of the first and second hems may comprise a longitudinally extending gathering member. The first fold line may form a proximal edge of the first side flap and the second fold line may form a proximal edge of the second side flap. The first and second proximal edges may be disposed laterally inward of the first and second side edges.

The first and second proximal edges of the first and second side flaps may be disposed between the longitudinal centerline and the first and second side edges, respectively.

The absorbent article may also comprise a second elastic gathering member attached at or adjacent the side edge of the absorbent article. Also, the absorbent article may comprise laterally opposing deployable belt ears attached to the web in at least one of the waist regions. Each belt ear may be disposed laterally inward until being deployed laterally outward so as to project laterally outward beyond the respective distal edges of the side flaps.

### BRIEF DESCRIPTION OF THE DRAWINGS

In the accompanying drawing figures, like reference numerals identify structurally corresponding elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description.

In the drawing figures and in the written description, lowercase letters appended to reference numerals indicate generally symmetric elements, e.g., left and right symmetric elements may be respectively identified by the reference numerals 1a and 1b. A reference numeral without an appended lowercase letter identifies all of the elements to which that particular reference numeral applies, e.g., the same elements as a group may be designated 1.

FIG. 1 is a plan view of an exemplary disposable absorbent article in the form of a diaper 20, which is shown in its flat, uncontracted state, i.e., without the contraction induced by elastic members. In this figure, the interior portion of the diaper 20 that faces inwardly toward the wearer and contacts the wearer is shown facing the viewer.

FIG. 2 is a plan view of the diaper 20 of FIG. 1 with the laterally opposing belt ears 108 extending laterally outward. In this figure, the interior portion of the diaper 20 is shown facing the viewer.

FIG. 3 is a plan view of the diaper 20 of FIG. 1 with the exterior portion of the diaper 20 shown facing the viewer.

FIG. 4 is a section view of the diaper 20 of FIG. 1 taken at the section line 4-4.

FIG. 5 is a section view of the diaper 20 of FIG. 1 taken at the section line 5-5.

FIG. 6 is a section view of the diaper 20 of FIG. 1 taken at the section line 6-6.

FIG. 7 is a section view of the diaper 20 of FIG. 1 taken at the section line 7-7.

FIG. 8 is a section view of an alternative embodiment of the diaper 20 of FIG. 1 taken at the section line 4-4.

FIG. 9 is a section view of an alternative embodiment of the diaper 20 of FIG. 1 taken at the section line 7-7.

FIG. 10 is a section view of an alternative embodiment of the diaper 20 of FIG. 2 taken at the section line 10-10 illustrating attached side flaps 147

FIG. 11 is a section view of the diaper 20 of FIG. 2 taken at the section line 11-11.

FIG. 12 is a section view of the diaper 20 of FIG. 2 taken at a section line similar to 12-12.

FIG. 13 is a perspective view of the diaper 20 of FIG. 1.

FIG. 14 is a section view of an alternative embodiment of the side flaps 147 of FIG. 1 taken at the section line 5-5.

FIG. 15 is a section view of an alternative embodiment of the side flaps 147 of FIG. 1 taken at the section line 5-5.

FIG. 16 is a section view of an alternative embodiment of the side flaps 147 of FIG. 1 taken at the section line 5-5.

FIG. 17 is a plan view of an alternative embodiment of the belt ears 108 of FIG. 1.

FIG. 18 is a section view of the diaper 20 of FIG. 17 taken at the section line 18-18.

FIG. 19 is the section view of the diaper 20 of FIG. 18 wherein the belt ears 108 are deployed laterally outward.

FIG. 20 is a section view of an alternative embodiment of the belt ears 108 of FIG. 17 taken at the section line 18-18.

FIG. 21 is a section view of an alternative embodiment of the belt ears 108 of FIG. 17 taken at the section line 18-18.

FIG. 22 is a plan view of exemplary belt ears 108 created from a single web via a nested cut line 92.

FIG. 23 is a plan view of an alternative embodiment of the belt ears 108 of FIG. 2 comprising nested belt ears 108.

FIG. 24 is a section view of an alternative embodiment of the belt ears 108 of FIG. 1 taken at the section line 7-7.

FIG. 25 is the section view of FIG. 24 wherein the belt ears 108 are deployed laterally outward.

FIG. 26 is a section view of an alternative embodiment of the belt ears 108 of FIG. 1 taken at the section line 7-7.

FIG. 27 is a section view of an alternative embodiment of the belt ears 108 of FIG. 1 taken at the section line 7-7.

FIG. 28 is a section view of an alternative embodiment of the belt ears 108 of FIG. 1 taken at the section line 7-7.

FIG. 29 is the section view of the diaper 20 of FIG. 28 wherein the belt ears 108 are deployed laterally outward.

FIG. 30 is a section view of an alternative embodiment of the belt ears 108 of FIG. 1 taken at the section line 7-7.

FIG. 31 is the section view of the diaper 20 of FIG. 30 wherein the belt ears 108 are deployed laterally outward.

FIG. 32 is a partial section view of an alternative embodiment of the belt ear 108 of FIG. 26 illustrating a peel bond, wherein the belt ear is multi-layered.

FIG. 33 is a partial section view of an alternative embodiment of the belt ear 108 of FIG. 27 illustrating a shear bond, wherein the belt ear is multi-layered.

FIG. 34 is a plan view of an alternative embodiment of exemplary backsheet 26, which is shown in its flat, uncontracted state, i.e., without the contraction induced by elastic members, and prior to formation of the side flaps. In FIG. 34, the exterior of the diaper is shown facing the viewer.

FIG. 35 is a cross section view of the diaper 20 of FIG. 34.

FIG. 36 is a plan view of an alternative embodiment of the backsheet 26 of FIG. 34.

FIG. 37 is a cross section view of the backsheet 26 of FIG. 36.

FIG. 38 is a plan view of an alternative embodiment of the backsheet 26 of FIG. 34.

FIG. 39 is a cross section view of the backsheet 26 of FIG. 38.

FIG. 40 is a plan view of an exemplary absorbent assembly 200 with the interior portion of the absorbent assembly 200 shown facing the viewer.

FIG. 41 is a section view of the absorbent assembly of FIG. 40 taken at the section line 41-41.

FIG. 42 is a view of the absorbent assembly of FIG. 40 taken at the section line 42-42.

FIG. 43 is a section view of an alternative embodiment of the absorbent assembly 200 of FIG. 40.

FIG. 44 is a section view of an exemplary storage component 272.

FIG. 45 is a section view of an alternative embodiment of the storage component 272 of FIG. 16.

FIG. 46 is a plan view of an exemplary disposable absorbent article in the form of a diaper 20, which is shown in its flat, uncontracted state, i.e., without the contraction induced by elastic members. In FIG. 46, the interior of the diaper is shown facing the viewer.

FIG. 47 is a section view of the diaper 20 of FIG. 46 taken at the section line 47-47.

FIG. 48 is a section view of the diaper 20 of FIG. 46 taken at the section line 48-48.

FIG. 49 is a section view of the diaper 20 of FIG. 46 taken at the section line 49-49.

FIG. 50 is a section view of the diaper 20 of FIG. 46 taken at the section line 50-50.

FIG. 51 is a section view of an alternative embodiment of the diaper 20 of FIG. 46 taken at the section line 48-48.

FIG. 52 is a section view of an alternative embodiment of the diaper 20 of FIG. 46 taken at the section line 48-48.

FIG. 53 is a section view of an alternative embodiment of the diaper 20 of FIG. 46 taken at the section line 48-48.

FIG. 54 is a section view of the inner liner 22 of FIG. 1 taken along the longitudinal axis 42.

FIG. 55 is a plan view of an exemplary disposable absorbent article in the form of a diaper 20, which is shown in its flat, uncontracted state, i.e., without the contraction induced by elastic members. In FIG. 55, the interior of the diaper is shown facing the viewer.

FIG. 56 is a section view of the diaper 20 of FIG. 55 taken at the section line 56-56.

FIG. 57 is a section view of the diaper 20 of FIG. 55 taken at the section line 57-57.

FIG. 58 is a section view of the diaper 20 of FIG. 55 taken at the section line 58-58.

FIG. 59 is a section view of the diaper 20 of FIG. 55 taken at the section line 59-59.

FIG. 60 is a plan view of an exemplary disposable absorbent article in the form of a diaper 20, which is shown in its flat, uncontracted state, i.e., without the contraction induced by elastic members. In FIG. 60, the interior of the diaper is shown facing the viewer.

FIG. 61 is a plan view of an exemplary fragment of a formed web material.

FIG. 62 is a plan view of an exemplary disposable absorbent article in the form of a diaper 20, which is shown in its flat, uncontracted state, i.e., without the contraction induced by elastic members. In this figure, the interior portion of the diaper 20 that faces inwardly toward the wearer and contacts the wearer is shown facing the viewer.

FIG. 63 is a section view of the diaper 20 of FIG. 62 taken at the section line 63-63.

FIG. 64 is a plan view of an alternative embodiment of an activation and attachment pattern 675 and 210 of FIG. 1, which is shown in its flat, uncontracted state, i.e., without the contraction induced by elastic members. In FIG. 64, the exterior of the diaper is shown facing the viewer.

FIG. 65 is a plan view of an alternative embodiment of an attachment pattern 210 of FIG. 1, which is shown in its flat, uncontracted state, i.e., without the contraction induced by elastic members. In FIG. 65, the exterior of the diaper is shown facing the viewer.

FIG. 66 is a plan view of an alternative embodiment of waist regions 36 and 38 of FIG. 1, wherein the waist regions 36 and 38 comprise apertures 622.

### DETAILED DESCRIPTION OF THE INVENTION

In this description, the following terms have the following meanings:

The term “absorbent article” refers to a device that absorbs and contains liquid, and more specifically, refers to a device that is placed against or in proximity to the body of a wearer to absorb and contain the various exudates discharged from the body.

The term “diaper” refers to an absorbent article that is generally worn by infants and incontinent persons about the lower torso so as to encircle the waist and the legs of the wearer and that is specifically adapted to receive and contain urinary and fecal waste. A diaper may be in the form of a taped diaper or may be pre-closed to form a pull-on (pant style) diaper.

The term “disposable” refers to the nature of absorbent articles that generally are not intended to be laundered or otherwise restored or reused as an absorbent article, i.e., they are intended to be discarded after a single use and may be recycled, composted or otherwise disposed of in an environmentally compatible manner.

The term “deploy” in all its forms refers to the manipulation of any disclosed deployable structural element from its initial configuration to a configuration in which it can be used for its intended purpose in the article on which it is provided.

The term “longitudinal” refers to a direction running from a waist edge to an opposing waist edge of the article and generally parallel to the maximum linear dimension of the article. Directions within 45° of the longitudinal direction are considered to be “longitudinal”.

The term “lateral” refers to a direction running from a side edge to an opposing side edge of the article and generally at a right angle to the longitudinal direction. Directions within 45° of the lateral direction are considered to be “lateral”.

The term “diagonal” refers to an orientation of a line extending obliquely relative to the longitudinal and lateral directions, i.e., neither perpendicular nor parallel to either of the longitudinal or lateral directions.

The term “circumferential” refers to a direction generally encircling the waist of the wearer parallel to the lateral direction. This term is used particularly when describing the elements that extend around and form the margin of the waist opening.

The term “disposed” refers to an element being attached and positioned in a particular place or position in a unitary structure with other elements.

The term “attach” refers to elements being connected or united by fastening, adhering, bonding, etc. by any method suitable for the elements being attached together and their constituent materials. Many suitable methods for attaching elements together are well-known, including adhesives, pressure bonding, thermal bonding, mechanical bonding, mechanical fastening, etc. Such attachment methods may be used to attach elements together over a particular area either continuously or intermittently. Unless indicated otherwise, elements that are described as being attached to each other are attached directly together, with either nothing or via one or more attachment members, e.g., an adhesive or a fastener, between them. Unless indicated otherwise, elements that are described as being attached to each other are attached permanently or temporarily together, i.e., permanently attached means that one or both of the elements and/or any attachment member that is present must be damaged in order to separate them. Temporary attached means that one or both of the elements and/or any attachment member that is present may be separated and reattached or refastened (i.e. open and closed) multiple times while substantially maintaining functionality of the attachment member.

The term “laminate” refers to elements being attached together in a layered arrangement.

The term “cohesive” refers to the property of a material that, once set, sticks to itself but does not to any significant degree stick to other materials.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

The terms “proximal” and “distal” refer respectively to the location of an element near to or far from the center of a structure, e.g., the proximal edge of a longitudinally extending element is located nearer to the longitudinal axis than the distal edge of the same element is located relative to the same longitudinal axis.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower”, “above” and “below”, “over” and “under”, and “top” and “bottom”, respectively.

The term “nonwoven” refers to a sheet, web, or batt of directionally or randomly oriented fibers, made by bonding or entangling the fibers through mechanical, thermal, or chemical means. Nonwoven materials exclude paper and products which are woven, knitted, tufted, or felted by wet milling. The fibers may comprise man-made synthetics.

The term “abdominal stretch panel” refers to a structural component that resists elongation by providing a laterally contractive force around the waist opening of a diaper when it is stretched in the lateral direction.

The term “application force” is the force required to extend the waist region of the diaper in order to apply the diaper onto the wearer.

The term “sustained fit force” is the force delivered by the diaper at the waist that provides the requisite body contact post application in order to deliver proper fit, gasketing, and sustained position (i.e., sustained fit).

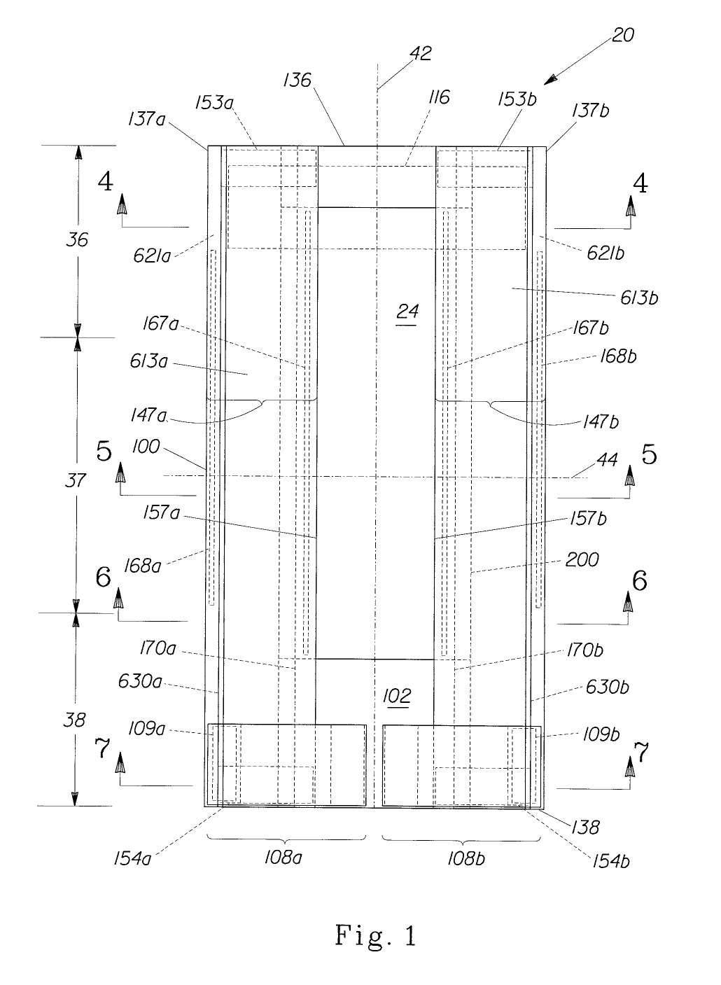

As shown in FIGS. 1, 2 and 3, one end portion of the exemplary diaper 20 may be configured as a front waist region 36, the longitudinally opposing end portion may be configured as a back waist region 38, and an intermediate portion may be configured as a crotch region 37.

The basic structure of the diaper 20 includes a chassis 100, which has a laterally extending front waist edge 136, a longitudinally opposing back waist edge 138, laterally opposing side edges 137, an interior surface 102, and an exterior surface 104. A longitudinal axis 42 extends through the midpoints of the front waist edge 136 and the back waist edge 138 and a lateral axis 44 extends through the midpoints of the side edges 137. The exemplary chassis 100 shown in FIG. 1 additionally has longitudinally extending and laterally opposing side flaps 147 as well as laterally opposing belt ears 108, which are described in more detail below. The portion of the chassis forming the backsheet and side flaps (including the side barriers and cuff flaps) may be formed by a web (herein, the “chassis web”) comprising one or more layers. One or more of the layers forming the chassis web 149 may be water impervious. The layers forming the chassis web 149 may have different lateral extents or may be coterminus in width. And, the chassis web 149, as well as the layer or layers forming the chassis web 149, may be laterally and/or longitudinally continuous.

The basic structure of the diaper 20 also includes an absorbent assembly 200 that may be attached to the interior surface of the chassis 100. The absorbent assembly 200 absorbs and retains liquid bodily waste materials. The absorbent assembly 200 has a laterally extending front edge 236, a longitudinally opposing back edge 238, laterally opposing side edges 237, an interior surface 202, and an exterior surface 204. The absorbent assembly 200 may be disposed either symmetrically or asymmetrically with respect to either or both of the longitudinal axis 42 and the lateral axis 44. For example, the absorbent assembly 200 shown in FIG. 1 is disposed symmetrically with respect to the longitudinal axis 42 and asymmetrically offset toward the front waist region 36 with respect to the lateral axis 44.

The edges of the absorbent assembly 200 may lie inward of the respective edges of the chassis 100, as in the exemplary diaper 20 shown in FIG. 1. Such a configuration in which one or more of the edges of the absorbent assembly 200 lies inward of the corresponding edges of the chassis 100 may be desirable, for example, in order to allow the relatively more flexible layer or layers adjacent to the edges of the chassis to conform to the body of the wearer and thereby form effective gasket-like seals against the skin of the wearer without being constrained by a relatively thicker and relatively less flexible absorbent assembly. Alternatively, one or more of the edges of the absorbent assembly 200 may coincide with the corresponding edge or edges of the chassis 100, e.g. the front edge 236 of the absorbent assembly may correspond to the front waist edge 136 of the chassis 100 and/or the back edge 238 of the absorbent assembly may correspond to the back waist edge 138 of the chassis. In certain embodiments, as few as one layer of the absorbent assembly, e.g. the upper covering sheet 24, may extend to and correspond with one or both of the front waist edge 136 or back waist edge 138 of the chassis.

In embodiments in which one or more of the layers of the absorbent assembly forming front edge 236 and/or back edge 238 of the absorbent assembly coincide with the respective front waist edge 136 or back waist edge 138 of the chassis and the side flaps 147 overlap the absorbent assembly 200, the side flaps 147 may be attached to the absorbent assembly 200 instead of, or in addition to, being attached to the interior surface 102 of the chassis 100.

In some embodiments, particularly those with a full length upper covering sheet 24, a portion of the absorbent assembly 200 disposed in one or both of the waist regions 36 and 38 may be laterally extensible to a greater extent than a potion of the absorbent assembly 200 in the crotch region 37. A portion of the absorbent assembly 200 may be rendered laterally extensible, for example, as described herein prior to being combined with the chassis 100 or in combination with the chassis 100 (as a whole diaper 20). Alternatively, for example, the chassis 100 and absorbent assembly 200 may be activated in one or both of the waist regions 36 and 38.

When the diaper 20 is worn on the lower torso of a wearer, the front waist edge 136, the laterally opposing belt ears 108 and the back waist edge 138 encircle the waist of the wearer, while at the same time the chassis side edges 137 encircle the legs of the wearer at least partially, the crotch region 37 is generally positioned between the legs of the wearer, and the absorbent assembly 200 extends from the front waist region 36 through the crotch region 37 to the back waist region 38.

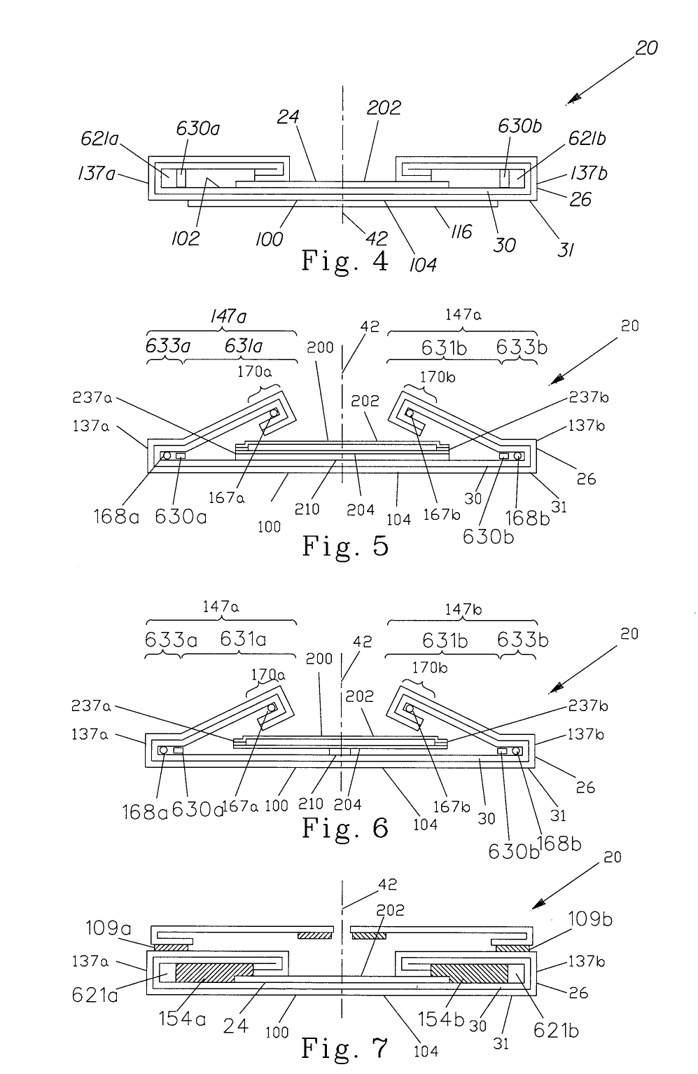

The chassis 100 includes a water-impermeable backsheet 26. The backsheet 26 forms an exterior surface that is intended to be placed toward any clothing that is worn over the diaper 20. Many suitable materials for use as the backsheet 26 are well-known, including films of polyethylene and other polyolefins. For example, the backsheet 26 may comprise a water vapor impermeable material or a water vapor permeable material. Exemplary materials suitable for use in the backsheet 26 include polyolefinic films, microporous or other breathable formed films, breathable monolithic films, and hydrophobic nonwovens. Multi-layer backsheets, such as a laminate of a film 30 and a nonwoven material 31 or a laminate of multiple nonwoven layers, may also be suitable for use as the backsheet 26. Such a backsheet may be oriented with the nonwoven 31 disposed exteriorly of the film, as shown in FIGS. 4, 5, 6 and 7, to provide the feel and appearance of a more cloth-like outermost layer than would be provided by using the film 30 as the outermost layer. A multi-layer backsheet 26, such as a laminate of a film 30 and a nonwoven 31, may also be suitable for use with the nonwoven 31 disposed interiorly to separate the film 30 from the skin of the wearer, or with nonwovens 31 disposed both exteriorly and interiorly such that the film is sandwiched. In embodiments in which the chassis 100 comprises a multi-layer backsheet 26, such as a laminate of a film 30 and a nonwoven 31, the nonwoven 31 may extend laterally to the full extent, width, of the backsheet 26, alternatively the nonwoven 31 may have a lateral extent that is less than that of the backsheet 26 or the nonwoven 31 may have a lateral extent that is greater than that of the backsheet 26.

In embodiments in which the chassis 100 comprises a multi-layer chassis web 149, such as a laminate of a film 30 and a nonwoven 31, the nonwoven 31 may extend laterally to the full extent, width of the film 30 or alternatively the nonwoven 31 may have a width that is less than or greater than that of the film 30 layer. In an embodiment where the nonwoven 31 has a width, lateral extent, that is less than that of the film 30, the portion of the chassis web 149 that is folded to form the side flap 147 may consist solely of the film 30 layer, or may consist substantially of the film 30 layer (in other words, for a multilayered chassis web 149, the nonwoven 31 may extend across the exterior surface 104 from one chassis side edge 137a to the opposing side edge 137b; or alternatively, the nonwoven 31 may wrap the chassis side edges 137a and b and cover a portion of the side flaps 147a and b). In an embodiment where the nonwoven 31 has a width, lateral extent, that is greater than that of the film 30, the portion of the chassis web 149 that is folded to form the side flap 147 may consist solely of the nonwoven 31 layer, or may consist substantially of the nonwoven 31 layer. In such an embodiment, the side flap 147 may comprise a single layer nonwoven 31 or may be folded to form a dual-layer nonwoven 31 side flap 147 that may be breathable.

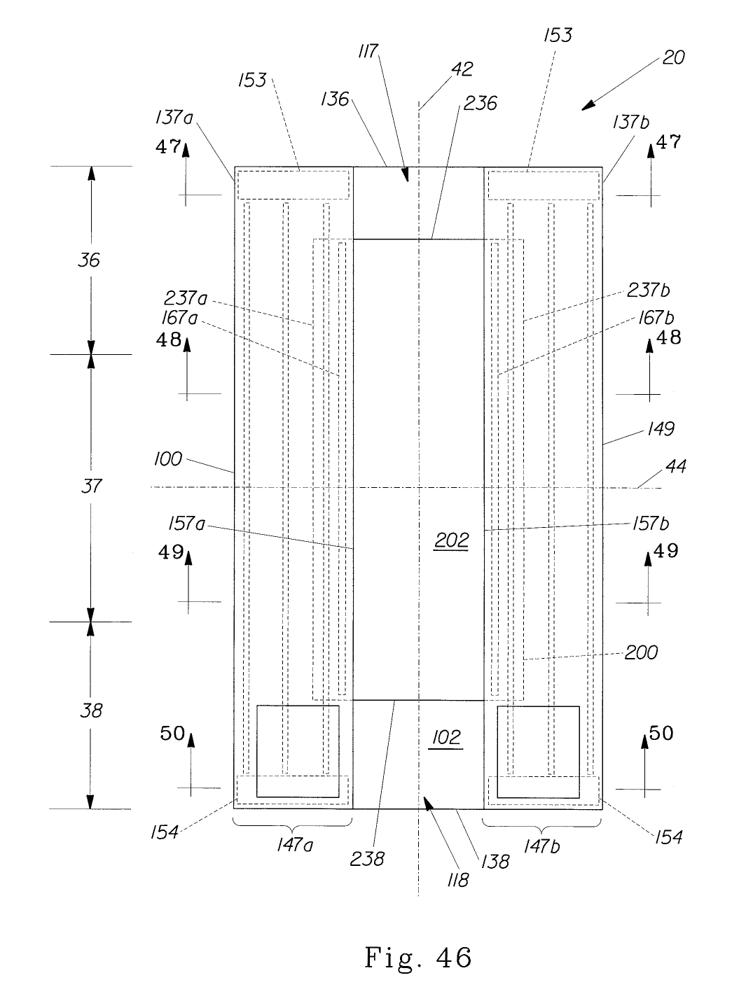

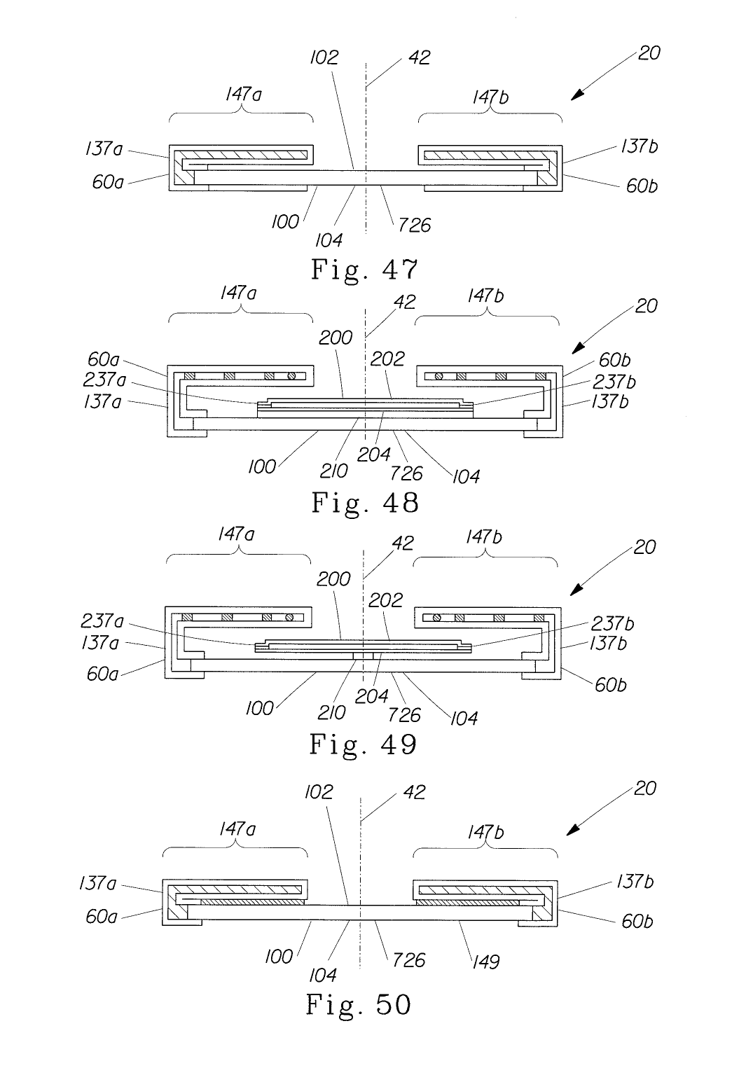

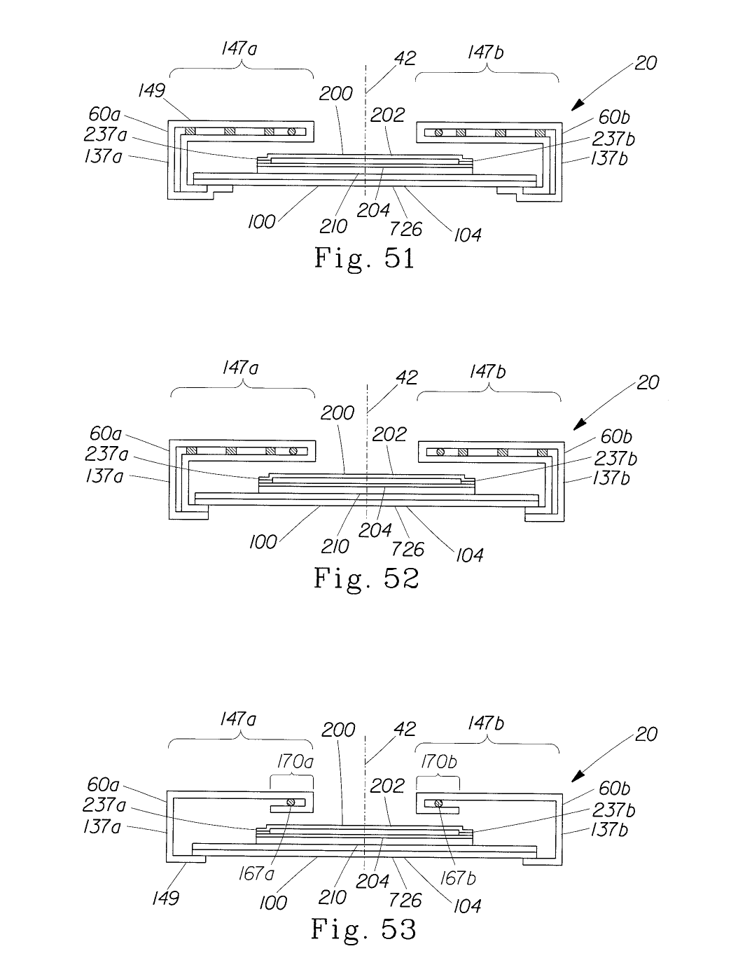

As shown in FIGS. 46-53, the chassis web 149 may be formed from multiple pieces, including a center sheet 726 and laterally opposing side sheets 60. The center sheet 726 and the side sheets may be formed from the same or different material types. For example, the center sheet 726 may comprise a water vapor impermeable material and the side sheets may comprise a water vapor permeable material. Exemplary materials suitable for use in the side sheets 60 include polyolefinic films, microporous or other breathable formed films, breathable monolithic films, and hydrophobic nonwovens. Suitable hydrophobic nonwovens include SM (spunbond meltblown), SMS (spunbond meltblown spunbond), and SMMS (spunbond meltblown meltblown spunbond) composites. The materials of the water vapor-permeable side sheets 60 may be selected to balance overall product economics and function. For example, a relatively more expensive nonwoven material having a relatively high basis weight may provide an acceptable level of water-impermeability for use in a single layer side flap construction. Alternatively, a relatively less expensive nonwoven having a relatively lower basis weight may provide the requisite level of water-impermeability only if it is doubled, thereby requiring a relatively greater area of material. As another example, a microporous film may provide a relatively optimal combination of water-impermeability and material cost. In a multi-layer chassis web 149 configuration one of the layers may comprise the center sheet 726 and side sheets 60 forming a first multi-piece layer. The first multi-piece layer may be attached to a separate layer which may comprise a single continuous material or may be in the form of a second multi-piece web. One such embodiment comprises a multi-piece film layer having a water vapor impermeable center sheet 726 and water vapor permeable side sheets. The multi-piece film layer may be laminated to a one piece nonwoven to form a multi-layer chassis web 149.

As shown in FIGS. 46-53, the exemplary chassis 100 has longitudinally extending and laterally opposing breathable side flaps 147a and 147b that are disposed on the interior portion of the diaper 20 that faces inwardly toward the wearer and contacts the wearer. The side flaps are formed by folding portions of the chassis 100 including the water vapor-permeable side sheets 60 laterally inward, i.e., toward the longitudinal axis 42, to form both the respective side flaps 147a and 147b and the side edges 137a and 137b of the chassis 100. The side sheets 60 of the chassis 100 may be folded laterally inward to form the side flaps. Alternatively, the chassis may be folded such that each side flap includes the respective side sheet 60 and a portion of the center sheet 726. In either configuration, at least a portion of each side flap is breathable due to its inclusion of at least a portion of the respective water vapor-permeable side sheet. Alternatively, the side sheet may be water vapor impermeable and the center sheet 726 may be water vapor permeable.

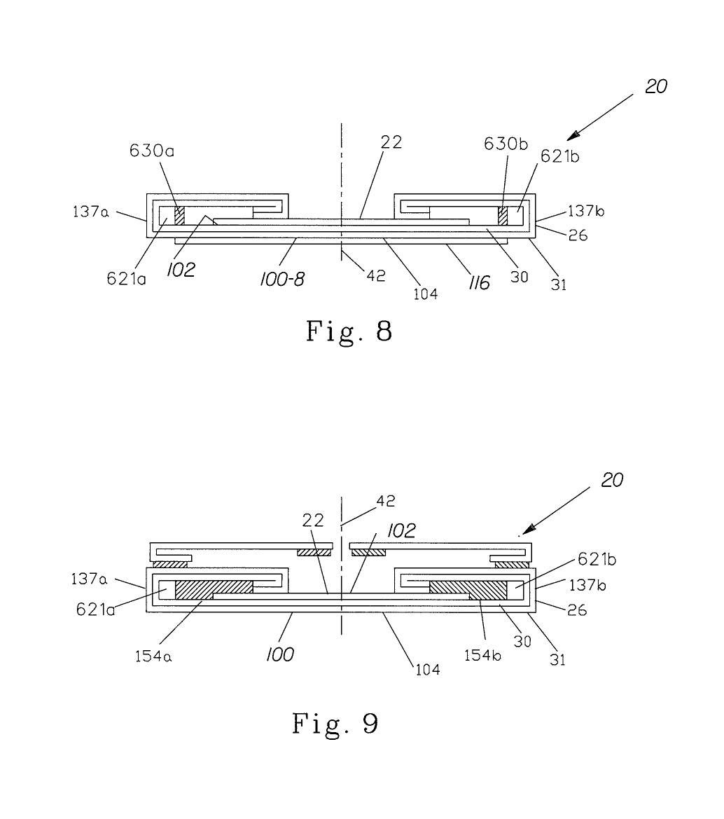

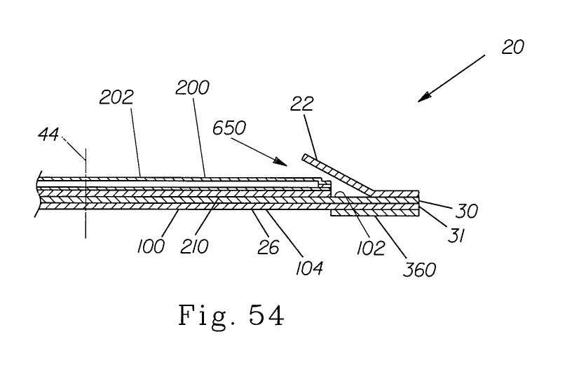

As illustrated in FIGS. 8 and 9, the chassis 100 may, but need not, additionally include an inner liner 22 attached to the backsheet 26. Such an inner liner 22 may be formed of a soft material that will not irritate the skin of the wearer. Many suitable materials for the inner liner 22 are well-known in the art, including rayon and synthetic nonwovens such as spunbonded or carded polypropylene, polyethylene, or polyester. An inner liner 22 may form a portion of the interior surface 102 of the chassis 100 such as in the respective front and back laterally central portions 117 and 118 between the edges 236 and 238 of, the absorbent assembly 200 and the waist edges 136 and 138 of the chassis 100 and thereby serve to isolate the skin of the wearer from a portion of the backsheet 26 as may be desirable, for example, when the diaper 20 is worn under conditions in which contact between the skin and a backsheet film could be uncomfortable. The inner liner 22 may be partially disposed under the side flaps 147 or alternatively the inner liner 22 may be disposed on top of the side flaps 147 in one or both of the waist regions 36 and 38. In certain embodiments, as shown in FIG. 54 the inner liner 22 may overlap a portion of the absorbent assembly 200. In such an overlapping configuration, it may be advantageous for the inner liner 22 to remain unattached to the absorbent assembly 200 over a portion of the longitudinal extent of the inner liner 22 particularly in the laterally central portions, area between the laterally opposing side flaps 147, for example 117 and 118, to provide an inner liner waist cap or stand up waist feature forming a pocket 650 which helps to prevent leakage of urine and/or feces at the waist.

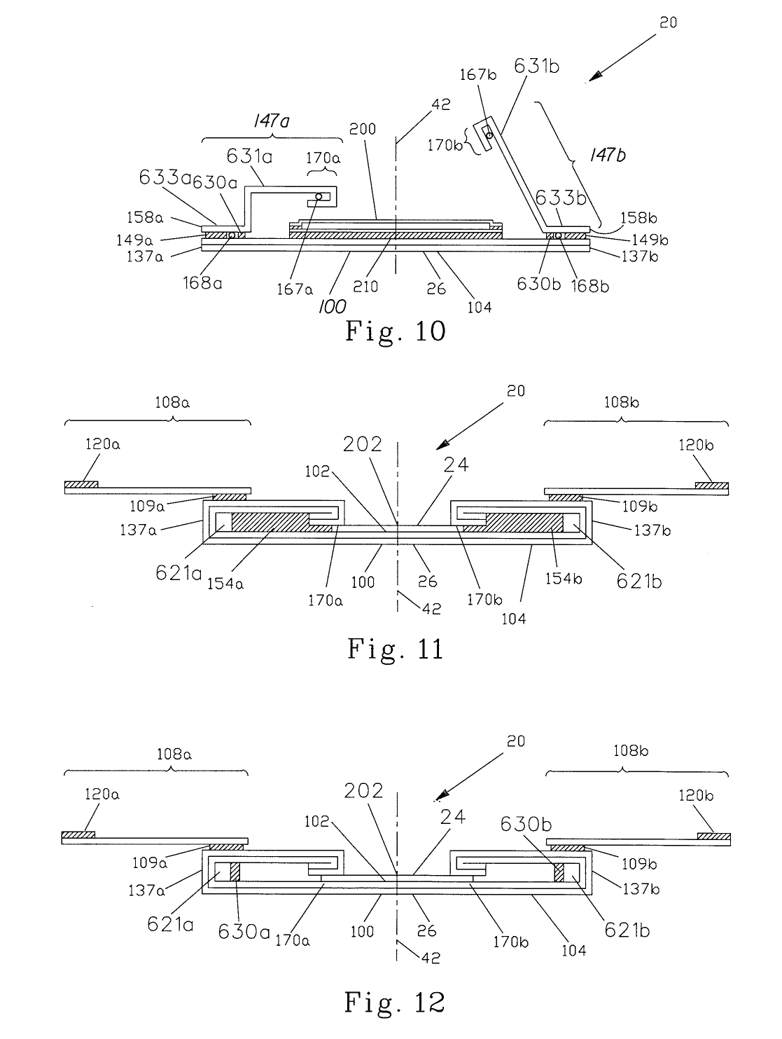

As shown in the figures, the exemplary chassis 100 has longitudinally extending and laterally opposing side flaps 147 that are disposed on the interior portion of the diaper 20 that faces inwardly toward the wearer and contacts the wearer. The side flaps 147 may be formed by folding portions of the chassis 100 laterally inward, i.e., toward the longitudinal axis 42, to form both the respective side flaps 147 and the side edges 137 of the chassis 100. Alternatively, as illustrated in FIG. 10, the side flaps 147 may be formed by attaching an additional layer or layers to the chassis 100 at or adjacent to each of the respective side edges 137 of the chassis 100. In embodiments in which the side flaps are formed by attaching an additional layer or layers to the chassis 100, each of the additional layer or layers may be attached in a distal edge attachment zone 149 at or adjacent to its laterally distal edge 158.

In the exemplary diaper 20 shown in FIG. 1, the side flaps 147 overlap the absorbent assembly 200, i.e., their proximal edges 157 lie laterally inward of the respective side edges 237 of the absorbent assembly 200. Such an overlapped configuration may be desirable in order to impart a more finished appearance to the diaper 20 than that imparted by a non-overlapped configuration. Alternatively, the side flaps 147 may not overlap the absorbent assembly 200.

Each side flap 147 may be attached to the interior surface 102 of the chassis 100 in a side flap attachment zone 153 adjacent to the front waist edge 136 and in a longitudinally opposing side flap attachment zone 154 adjacent to the back waist edge 138, as shown in the figures.

Between the side flap attachment zones 153 and 154, the proximal edge 157 of the side flap 147 remains free, i.e., not attached to the interior surface 102 of the chassis 100 or to the absorbent assembly 200. Also between the longitudinally opposing side flap attachment zones, each side flap may include one or more (specifically including one, two, three, or four strands per side flap) longitudinally extensible flap elastic gathering members that may be attached adjacent to the proximal edge of the side flap by any of many well-known means. Each of such flap elastic gathering members may be attached over its entire length or over only a portion of its length. For example, such flap elastic gathering members may be attached only at or near its longitudinally opposing ends and may be unattached at the middle of its length. Such flap elastic gathering members may be disposed in the crotch region 37 and may extend into one or both of the front waist region 36 and the back waist region 38. For example, in the exemplary chassis 100 shown in FIG. 1, an elastic strand 167 may be attached adjacent to the proximal edge 157 of each of the side flaps 147 and extends into both the front waist region 36 and the back waist region 38.

Each flap elastic gathering member may be enclosed inside a folded hem. For example, in the exemplary chassis 100 shown in FIG. 4 and FIG. 5, each of the elastic strands 167 may be enclosed inside a hem 170 formed adjacent to the proximal edge 157 of the respective side flap 147. Alternatively, the flap elastic gathering member(s) may be sandwiched between two layers of the chassis or may be attached on a surface of the chassis 100 and remain exposed.

When stretched, the flap elastic gathering member disposed adjacent to each side flap proximal edge allows the side flap edge to extend to the flat uncontracted length of the chassis, e.g., the length of the chassis 100, as shown in FIG. 1. When allowed to relax, the flap elastic gathering member contracts to pull the front waist region 36 and the back waist region 38 toward each other and thereby bend the diaper 20 into a “U” shape in which the interior of the “U” shape may be formed by the portions of the diaper 20 that are intended to be placed toward the body of the wearer. Because each of the proximal edges 157 remains free between the longitudinally oriented side flap attachment zones 151 and 152, the contractive force of the elastic strand 167 lifts the proximal edge 157 of the side flap 147 away from the interior surface 102 of the chassis 100. This lifting of the proximal edges 157 when the diaper 20 is in the relaxed condition lifts the side flaps 147 into position to serve as side barriers adjacent to the side edges 237 of the absorbent assembly 200.

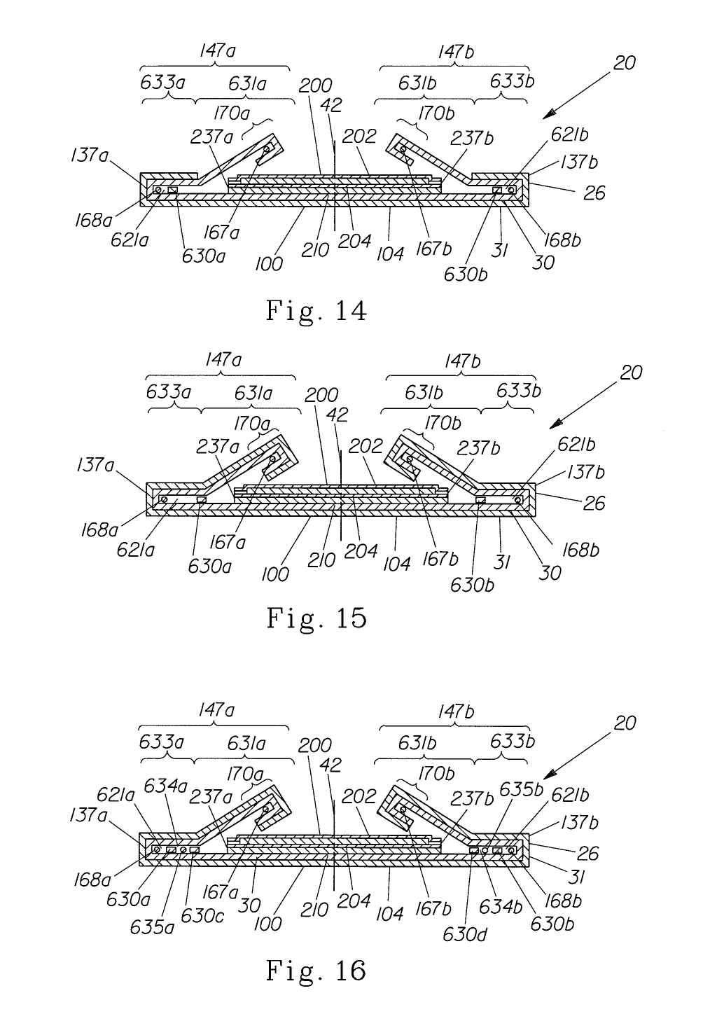

As shown in FIGS. 5, 6, 10, and 14-16, one or more (specifically including one, two, three, or four strands per side flap) second elastic strands 168a and b may be attached at or adjacent the side edge 137a and b of the chassis 100 where it maybe folded to form the side flap 147a and b. When allowed to relax, the second elastic strands 168a and b may gather the side edges 137a and b of the chassis 100 to form side barriers 633a and b and function as a barrier to leakage of urine and fecal waste. A channel 621a and b may be formed adjacent the side edge 137a and b of the chassis 100 where it may be folded to form the side flap 147a and b such that the portion of the chassis web 149 forming the channel 621a and b remains largely non-adhered to itself or to the second elastic strand 168a or b, particularly in each of the opposing waist regions 36 and 38, such that the second elastic strand 168a and b floats in the hollow of the channel 621a and b to enable the second elastic strand 168a and b to snap back to its glued-in length once the diaper 20 is cut to length during manufacture.

The side flap 147 may be formed into a cuff flap 631 and a side barrier 633. Particularly, side barrier attachment zone 630 may be oriented between the first and second elastic strands 167a and b and 168a and b. The placement of side barrier attachment zones 630a and b relative to the longitudinal axis 42 has a direct and coupled effect on the depth of cuff flaps 631a and b and the size of the side barriers 633a and b. For example, as illustrated by FIG. 15, when the side barrier attachment zone 630 is moved laterally inward, the depth of the cuff flaps 631 decreases and the size of the side barrier 633 increases. Conversely, when the side barrier attachment zone 630 is moved laterally outward, the depth of the cuff flaps 631 increases and the size of the side barriers 633 decreases. The depth and/or size of the cuff flaps 631 and side barriers 633 may be adjusted for various applications to provide enhanced functionality. In one such embodiment, it has been found that reduced depth cuff flaps 631 and larger side barriers 633 provides better application ease with regard to a pull-on pant style application. This configuration increases the size of the leg opening 12 enabling the wearer to step into the diaper 20 more easily. In yet another embodiment, it has been found that increasing the depth of the cuff flaps 631 and reducing the size of the side barriers 633 provides improved leakage protection and increased perception of capacity.

The depth of the cuff flaps 631 as measured from the proximal edge of the side flaps 147 to the side barrier attachment zones 630 may be from about 2 cm to about 7 cm, from about 2.5 cm to about 6.5 cm, or from about 3 cm to about 6 cm. The length of the side flaps 147, as measured from the proximal edge of the side flap 147 to the side edge of the chassis 137 may be from about 4.5 cm to about 9.5 cm, from about 5 cm to about 8.5 cm, or from about 5.5 cm to about 7.5 cm. Alternatively, as shown in FIG. 16, a second pair of side barrier attachment zones 630 may be used such that one can adjust the height of the cuff flaps 631 portion of the side flaps 147 without impacting the height of the barrier cuffs 633. A channel 634a and b may be formed between side barrier attachment zones 630a and c and/or 630b and d and may comprise an elastic strand 635a and b disposed therein.

For embodiments wherein the side flaps 147 are formed by attaching additional layers to the chassis 100 at or adjacent to each of the respective side edges 137a and b of the chassis 100, the second elastic strand 168a and b may be oriented and attached between the layers (e.g., layers forming the backsheet 26 and side flap 147) as illustrated in FIG. 10. The layers may also form a channel (e.g., 621a and b) and provide the functionality associated with it as described above.

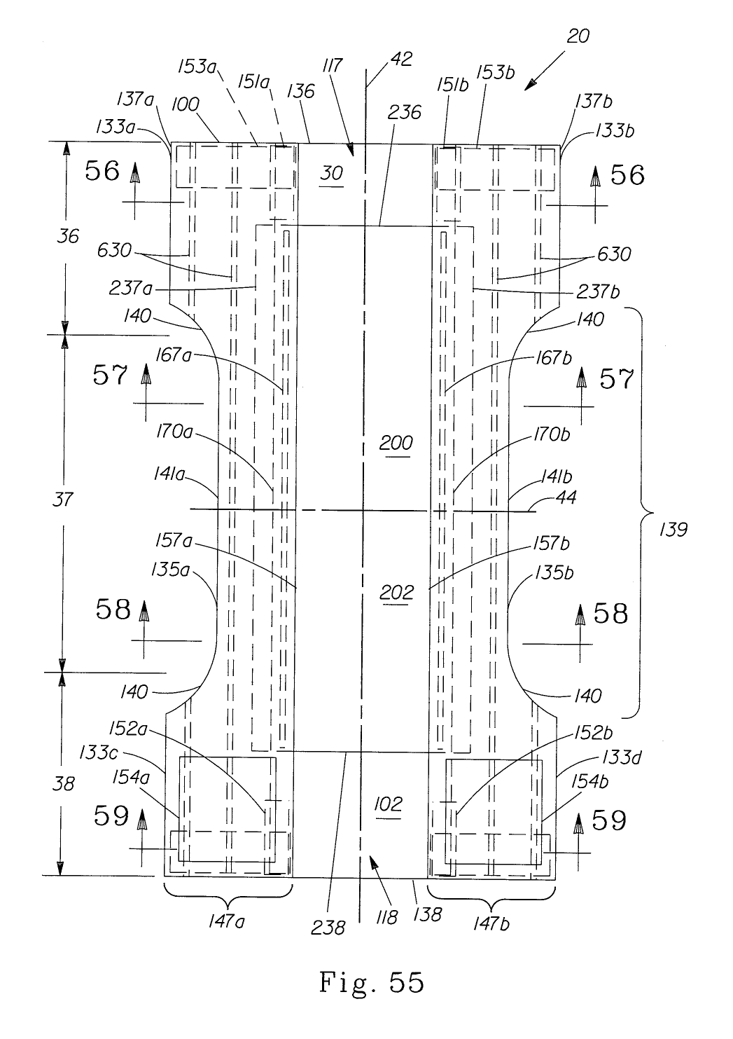

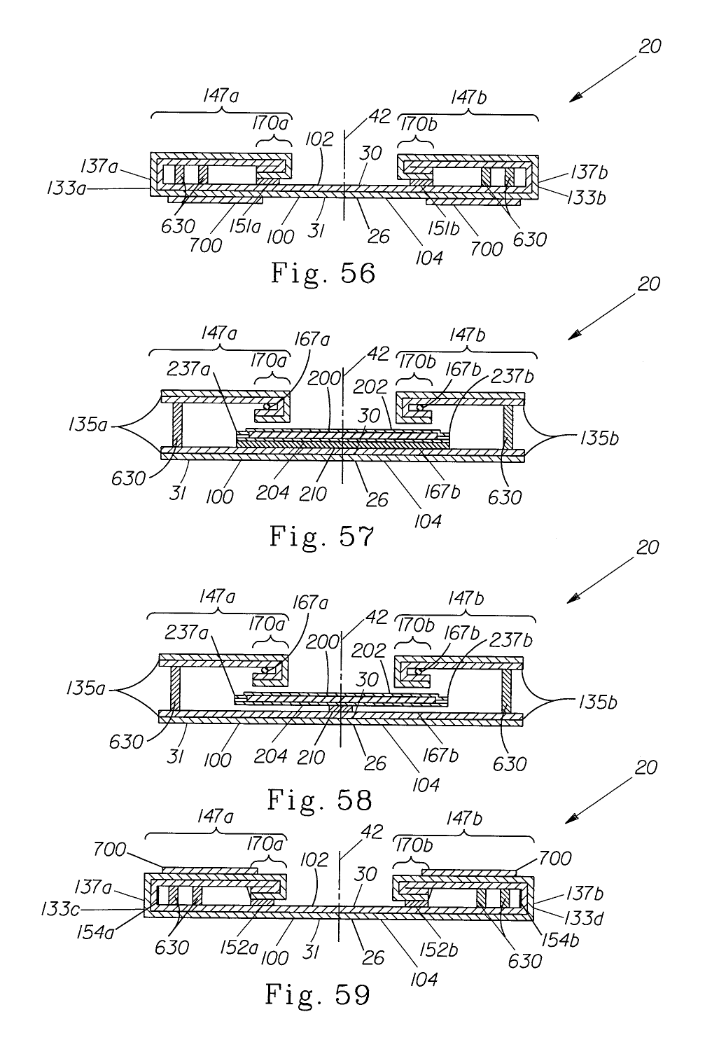

As shown in FIGS. 55-60, the basic structure of the diaper 20 includes a chassis 100. The chassis 100 has a laterally extending front waist edge 136 in the front waist region 36 and a longitudinally opposing and laterally extending back waist edge 138 in the back waist region 38. The finished chassis 100 has longitudinally extending front folded side edge segments 133a and 133b in the front waist regions, longitudinally opposing longitudinally extending back folded side edge segments 133c and 133d in the back waist region, and longitudinally extending cut side edge segments 135 in at least the crotch region, each of the cut side edge segments 135 connecting the respective front and back folded side edge segments 133. In combination, the respective folded side edge segments 133 and cut side edge segments 135 form the composite side edges 137, which connect the front waist edge and the back waist edge. The chassis 100 has an interior surface 102 and an exterior surface 104. The chassis 100 also has a longitudinal axis 42 and a lateral axis 44. The longitudinal axis 42 extends through the midpoint of the front waist edge 136 and through the midpoint of the back waist edge 138. The lateral axis 44 extends through the midpoint of the left side edge 137a and through the midpoint of the right side edge 137b. The exemplary chassis 100 shown in FIG. 55 additionally has longitudinally extending and laterally opposing side flaps 147a and 147b that are described in more detail below.

In the finished diaper, the chassis has a generally “hourglass” shape, as in the exemplary diaper 20 shown in FIG. 55. Such a non-rectangular shape may be desirable in order to impart a tailored appearance to the diaper 20 when it is worn. Such a non-rectangular shape may also be desirable in order to impart an impression that the diaper 20 will fit comfortably between the legs of a wearer.

The chassis 100 is given the hourglass shape by the removal of laterally opposing portions of the chassis 100 from at least the crotch region 137, to form laterally opposing side notches 139. This formation of the side notches 139 in the chassis makes its lateral dimension at and adjacent to the lateral axis 44 smaller than its lateral dimension at and adjacent to the front waist edge 136 and smaller than its lateral dimension at and adjacent to the back waist edge 138, i.e., makes the chassis narrower in the crotch region 37 than at the waist edges 136 and 138.

The contour of the side notch 139 that is formed by the removal folded material along the cut side edge segment 135 is defined by the contour of that cut side edge segment. The contour may be continuously arcuate. Alternatively, the contour may be a composite formed by two longitudinally opposing arcuate portions 140 and a generally straight intermediate portion 141 connecting the arcuate portions, and thus not continuously arcuate, as in the exemplary diapers 20 shown in FIG. 55. The side notches 139 may be disposed symmetrically with respect to either or both of the longitudinal axis 42 and the lateral axis 44 of the chassis 100. For example, the side notches 139 are shown disposed symmetrically with respect to both the longitudinal axis 42 and the lateral axis 44 in FIG. 55. Alternatively, the side notches 139 may be disposed asymmetrically with respect to either or both of the longitudinal axis 42 and the lateral axis 44. For example, the side notches 139 may be disposed symmetrically with respect to the longitudinal axis 42 and asymmetrically with respect to the lateral axis 44, for example where the side notches are offset toward the front waist edge 136.

The formation of the side notches 139 by the removal of the laterally opposing portions of the chassis leaves only longitudinally separated segments of the folded side edges 133 intact to prevent any bodily waste material from migrating laterally and escaping from the diaper 20. In particular, only the front folded side edge segments 133a and 133b and the back folded side edge segments 133c and 133d remain intact. The removal of the side portion 142 to form each side notch 139 detaches the respective side flap 147 from the underlying layer of the chassis along the contour of each cut side edge segment 135, thus creating an opening through which bodily waste material could escape. Therefore, the chassis 100 includes at least one longitudinally extending continuous barrier attachment 630 disposed laterally proximally of each cut side edge segment 135. Each side flap 147 is attached to the underlying layer of the chassis 100 at the barrier attachment 630. Each barrier attachment 630 is water-impermeable at least laterally and thereby preferably acts as a dam to prevent the lateral escape of bodily waste material in the gap between the longitudinally separated front and back folded side edge segments.

A single barrier attachment 630 may be used to attach each side flap 147 or, alternatively, two or more barrier attachments 630 may be used to attach each side flap 147. One or more barrier attachments 630 used to attach each side flap can be described as forming a barrier attachment zone (that is, each side flap having its own barrier attachment zone). For example, in the exemplary diaper 20 shown in FIG. 55, two laterally spaced barrier attachments 630 may be used to attach each side flap 147. As another example, a single barrier attachment 630 may be used to attach each side flap 147 in the exemplary diapers 20. As yet another example, four laterally spaced barrier attachments 630 may be used to attach each side flap 147 in the exemplary diaper 20. The use of a single barrier attachment 630 for each side panel 147 may help to minimize the cost of the diaper 20. On the other hand, the use of more than one barrier attachment 630 for each side panel 147 may help to prevent the lateral escape of bodily waste materials in the event that one side seal is not perfectly continuous and thereby allows flow past itself.

The barrier attachments 630 may be oriented generally parallel to the longitudinal axis 42 and to each other, as shown in FIG. 55. The barrier attachments 630 may be configured as mirror images of each other.

The barrier attachments 630 may be formed by heat bonding, pressure bonding, a combination of heat bonding and pressure bonding, ultrasonic bonding, adhesive bonding, or in any other way or combination of ways known in the art for forming laterally water-impermeable bonds. Each barrier attachment 630 may extend from the front waist edge 136 to the back waist edge 138, as shown in FIG. 55. Alternatively, a barrier attachment 630 may extend less far in the longitudinal direction. For example, a barrier attachment 630 may extend longitudinally only as far as the respective cut side edge segment 135 extends.

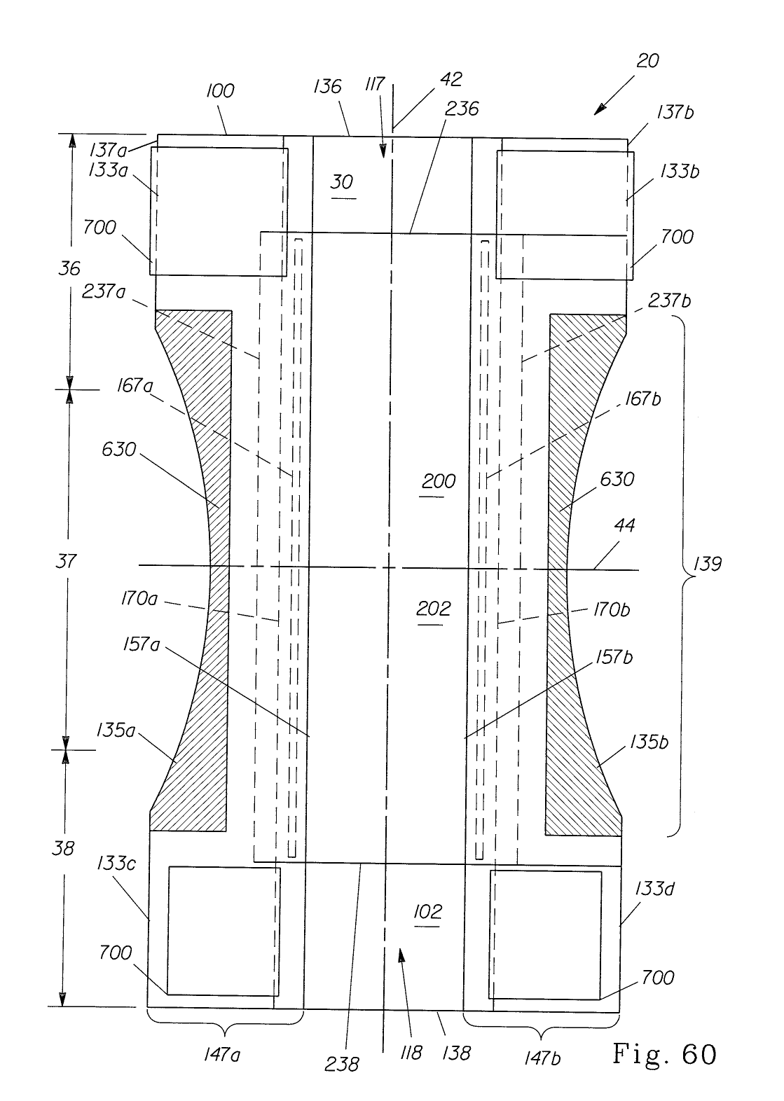

The barrier attachment 630 may initially extend through the side portion of the chassis 100 that is eventually removed to form the side notch 139. Exemplary barrier attachments 630 remaining longitudinally continuous after the formation of the side notches 139 are also shown in the finished exemplary diaper 20 of FIG. 55 and FIG. 60.

The barrier attachments 630 may be substantially linear in form as shown in FIG. 55. Alternatively, a barrier attachment 630 may be curvilinear in form. For example, a barrier attachment 630 may have a contour generally concentric to the contour of the cut side edge segment 135 and thereby “follow” the contour of the cut side edge segment from a point at or adjacent to a respective front folded side edge segment 133a or 133b to the respective corresponding back folded side edge segment 133c or 133d. In such a configuration, the combination of the front folded side edge segment, the side seal, and the back folded side edge segment may form a longitudinally continuous barrier to the lateral flow of bodily waste material between the front and back waist edges. As another alternative in which a combination of the front folded side edge segment, the side seal, and the back folded side edge segment may form a longitudinally continuous barrier to the lateral flow of bodily waste material, a barrier attachment 630 may be sufficiently wide to extend from laterally inward of the cut side edge segment 135 to the folded side edge segments 133, such as the exemplary side seals shown in FIG. 60.

Each barrier attachment 630 may be formed as a relatively narrow stripe being at least ten times as long as it is wide, such as the exemplary side seals shown in FIG. 55. Alternatively, each barrier attachment 630 may be formed as a relatively wide band being less than ten times as long as it is wide, such as the exemplary side seals shown in FIG. 60.

A portion or the whole of the chassis 100 may be made extensible to a degree greater than the inherent extensibility of the material or materials from which the chassis is made, e.g., the backsheet 26. The additional extensibility may be desirable in order to allow the chassis 100 to conform to the body of a wearer during movement by the wearer. The additional extensibility may also be desirable, for example, in order to allow the user of a diaper 20 including a chassis 100 having a particular size before extension to extend the front waist region 36, the back waist region 38, or both waist regions of the chassis 100 to provide additional body coverage for wearers of differing size, i.e., to tailor the diaper to the individual wearer. Such extension of the waist region or regions may give the diaper a generally hourglass shape, so long as the crotch region 37 is extended to a relatively lesser degree than the waist region or regions, and may impart a tailored appearance to the diaper 20 when it is worn. In addition, the additional extensibility may be desirable in order to minimize the cost of the diaper. For example, an amount of material that would otherwise be sufficient only to make a relatively smaller diaper lacking this extensibility can be used to make a diaper capable of being extended to adequately cover a wearer that is larger than the non-extended, smaller, diaper would fit.

A portion of the chassis 100 in the front and/or back waist regions 36 and 38 may be made laterally extensible to a maximum extensibility greater than a maximum extensibility of another portion of the chassis 100 in the crotch region 37 such that a lateral extension of each of the portions in the front, back, and crotch regions 36, 38, and 37 to its maximum extensibility imparts an hourglass shape to the chassis 100. As disclosed in U.S. patent Ser. No. 12/358,962, filed on Jan. 23, 2009, titled, Extrusion Bonded Laminates for Absorbent Articles, one or more layers of the chassis web may comprise an elastomeric film which may provide the desired amount of extension and recovery forces during use of the laminate. As mentioned above, lateral extensibility may be achieved by incorporating an elastomeric film into one or more layers of the chassis web. Many suitable elastic materials that may be used for one or more layers of the chassis web include synthetic or natural rubbers (e.g., crosslinked polyisoprene, polybutadiene and their saturated versions (after hydrogenation), and polyisobutylene), thermoplastic elastomers based on multi-block copolymers, such as those comprising copolymerized rubber elastomeric blocks with polystyrene blocks (e.g., styrene-isoprene-styrene, styrene-butadiene-styrene, styrene-ethylene/butylene-styrene, styrene-ethylene/propylene-styrene, and styrene-butadiene/isoprene-styrene, including their hydrogenated and non-hydrogenated forms), thermoplastic elastomers based on polyurethanes, polyesters, polyether amides, elastomeric polyolefins including polyethylenes and polypropylenes, elastomeric polyolefin blends, and combinations thereof.

For instance, one useful group of elastomeric polymers that may be used in the chassis web are the block copolymers of vinyl arylene and conjugated diene monomers, such as AB, ABA, ABC, or ABCA block copolymers where the A segments may comprise arylenes such as polystyrene and the B and C segments (for those embodiments comprising B and/or C segments) may comprise dienes such as butadiene or isoprene. A similar, newer group of elastomeric polymers are the block copolymers of vinyl arylene and hydrogenated olefin monomers, such as AB, ABA, ABC, or ABCA block copolymers where the A segments may comprise arylenes such as polystyrene and the B and C segments (for those embodiments comprising B and/or C segments) may comprise saturated olefins such as ethylene, propylene, or butylene. Suitable block copolymer resins are readily available from KRATON® Polymers of Houston, Tex., Dexco™ Polymers LP of Planquemine, La., or Septon™ Company of America, Pasadena, Tex.

Another useful group of elastomeric polymers that may be used in the chassis web are olefin-based elastomers. In one embodiment, the elastomeric film comprises a polyolefinic elastomer (POE). Examples of POEs include olefin block copolymers (OBCs) which are elastomeric copolymers of polyethylene, sold under the trade name INFUSE™ by The Dow Chemical Company of Midland, Mich. Other examples of POEs include copolymers of polypropylene and polyethylene, sold under the trade name VISTAMAXX® by ExxonMobil Chemical Company of Houston, Tex. and/or VERSIFY by Dow Chemical, Midland, Mich.

For the elastomeric film portion of the chassis web, other polymers may be blended into the compositions to enhance desired properties. For example, a linear low-density polyethylene may be added to the film composition to lower the viscosity of the polymer melt and enhance the processability of the extruded film. High-density polyethylene may be added to prevent age-related degradation of the other polymers. Polypropylene has been found to improve the robustness of the elastomer and improve the films' resistance to pinholing and tearing. Additionally, polypropylene-based thermoplastic elastomer reactor blends (e.g., ADFLEX, available from LyondellBasell Industries, Laporte, Tex.) may be used to increase the toughness the film, as disclosed in WO 2007/146149.

Additional lateral extensibility in the chassis 100 or portions of the absorbent assembly 200 may be provided in a variety of ways as describe hereinafter. For example, a material or materials from which the chassis 100 is made may be pleated by any of many known methods. Alternatively, all or a portion of the chassis may be intermittently activated to create a structured elastic-like formed web material or a formed laminate of web materials like those described in U.S. Pat. No. 5,518,801 issued on 21 May 1996 in the name of Chappell et al. This formed web material includes distinct laterally extending regions in which the original material has been altered by embossing or another method of deformation to create a pattern of generally longitudinally oriented alternating ridges and valleys and also includes laterally extending unaltered regions between the laterally extending altered regions. The formed web material can be extended in a direction perpendicular to the ridges up to the point where the ridges and valleys flatten with substantially less force than is required to extend beyond that point. In addition to lateral extensibility, the creation of a formed laminate web as described above provides the backsheet 26 with improved texture and cloth-like appearance and feel. The deformation creates a cloth-like pattern in the film and increases the loft of the nonwoven 31 in multi-layer film 30 and nonwoven 31 laminate backsheets 26.

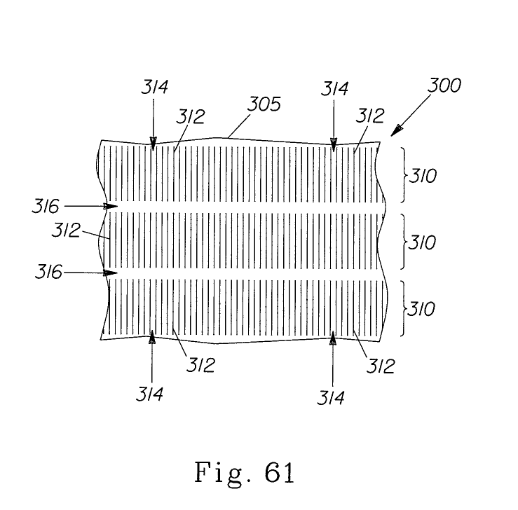

An exemplary fragment 300 of such a formed web material 305 is shown in FIG. 61. This formed web material 305 includes distinct laterally extending regions 310 in which the original material has been altered by embossing or another method of deformation to create a pattern of generally longitudinally oriented alternating ridges 312 and valleys 314. The formed web material 305 also includes laterally extending unaltered regions 316 located between the laterally extending altered regions 310.

Such a formed web material 305 can be laterally extended beyond its original dimension with the application of relatively less force than that required to extend the same material to the same extent when undeformed. In particular, the effects of an application of opposing divergent forces directed generally perpendicular to the ridges 312 and valleys 314 include an extension of such a formed web material along an axis between the opposing forces and the generation of a resistive contractive force, primarily in the unaltered regions 316. This resistive force is relatively smaller than the resistive force that is generated by the same material in its unaltered form when extended to the same extent, at least up to an extension at which the ridges and valleys in the altered regions flatten and begin to contribute to the resistive force. Thus, such formed web materials exhibit an extensible behavior resembling that of traditional elastic materials in the range of extensibility that is useful for the type of lateral extension desired for use in absorbent articles. However, such formed web materials may be made of relatively less expensive materials that are not inherently elastic and, thus, their use may provide an advantage in terms of the cost of manufacturing the absorbent articles.

The activation approaches described above are achieved by using a set of opposing rolls comprising a staggered orientation of teeth such that when a material such as the chassis web 149 or backsheet passes through the intermeshing rolls, the material is deformed to create the desired activation pattern in the web. In one embodiment, the teeth of one of the rolls may be continuous around the circumference of the roll while the teeth on the opposing roll may be discontinuous forming intermittent activation (e.g. a structured elastic-like formed web) or region specific activation (e.g. activation in the opposing waist regions and not the crotch region). The depth to which the teeth intermesh, i.e. depth of engagement, will determine to what degree the web is incrementally stretched and/or plastically deformed and therefore the degree of extensibility imparted to the web.

In some embodiments, the absorbent article may be activated in a specific region, e.g. front and/or back waist region, to a greater extent than an adjacent region. For example, one or both of the waist regions of the chassis may comprise a material that has been deformed to a greater extent forming larger (i.e. deeper) longitudinally oriented ridges and valleys while other portions of the absorbent article may comprise relatively smaller (i.e. shallower) longitudinally oriented ridges and valleys.

The front laterally central portion 117 and the back laterally central portion 118 of the chassis 100 may have a different range of extensibility from the portions of the chassis in the side flap attachment zones 153 and 154 where the side flaps 147 are attached. Additionally or alternatively, the laterally central portions 117 and 118 may be extensible to a greater or lesser degree when subjected to a given level of opposing tensile forces, i.e., may be more easily or less easily extensible, than the portions of the chassis in the side flap attachment zones 153 and 154. For example, if the chassis is made uniformly extensible across its entire width prior to the formation of the side flaps, the double layering in the areas of the side flap attachment zones 153 and 154 after the formation of the side flaps may have an effect of decreasing the degree of lateral extensibility of those areas under a given level of opposing tensile forces, such as by the side flaps acting as parallel “springs” that must be extended in order to extend the underlying attached portion of the chassis. As another example, the altered regions in the laterally central portions of the chassis may be deformed to a greater or a lesser degree than the altered regions in the side flap attachment zones 153 and 154 to render the laterally central portions more easily or less easily extensible than the respective portions in the side flap attachment zones 153 and 154.

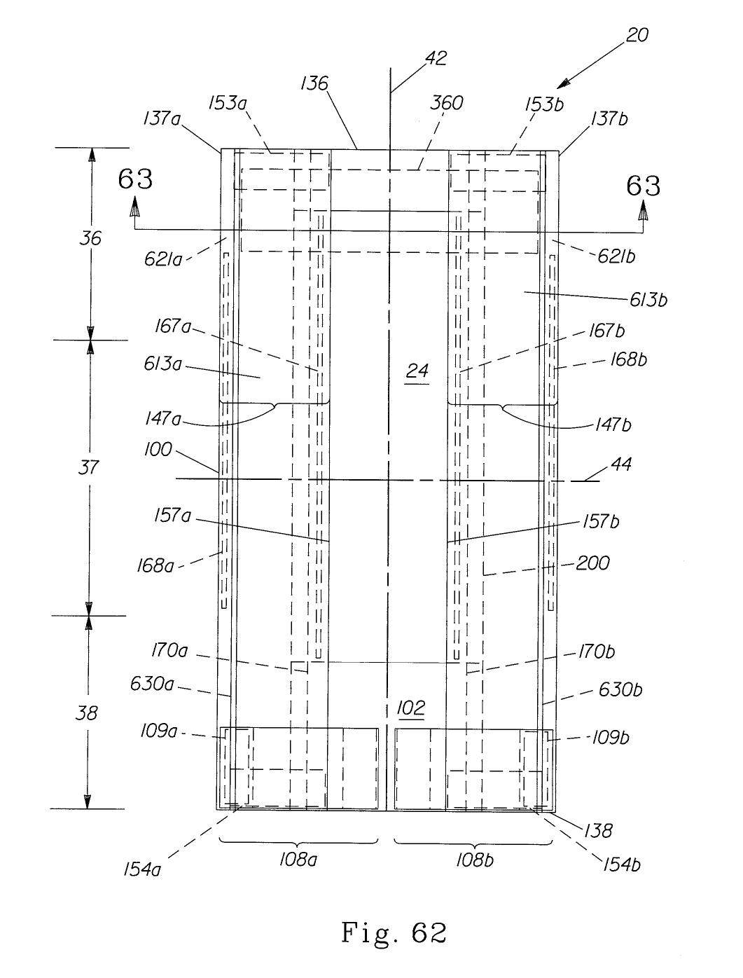

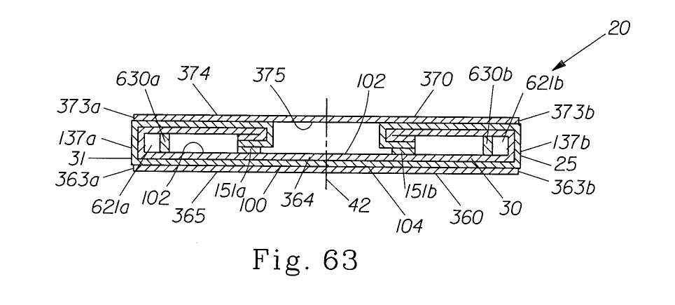

As shown in FIGS. 62 and 63, the diaper may also comprise one or more abdominal stretch panels having a laterally extending longitudinally distal edge that is disposed at or adjacent to the respective waist edge of the chassis 100 and a longitudinally opposing laterally extending longitudinally proximal edge that is disposed relatively nearer to the lateral axis 44 than the longitudinally distal edge of the same abdominal stretch panel is disposed. For instance, the front and back waist regions may each comprise an abdominal stretch panel. In some embodiments of the present invention, the front and back waist regions may each comprise a plurality of abdominal stretch panels. The chassis may comprise one or more abdominal stretch panels disposed on the interior surface of one or a combination of the absorbent assembly, backsheet, side barriers and cuff flaps and the exterior surface of the backsheet. Each abdominal stretch panel also has laterally opposing longitudinally extending side edges, an interior surface, and an exterior surface.

The abdominal stretch panels may have a lateral extent that is substantially equivalent to the lateral extent of the chassis 100 in the respective waist region. Alternatively, the abdominal stretch panels may have a lateral extent that is less than the lateral extent of the chassis 100 in a particular waist region. For example, an interior abdominal stretch panel may have a lateral extent only approximately as great as the distance between the proximal edges 157 of the side flaps 147. As another example, an interior abdominal stretch panel may have a lateral extent greater than the distance between the proximal edges 157 of the side flaps 147 but less than the distance between the side edges 137 of the chassis 100. The portions of such an interior abdominal stretch panel that lie laterally outboard of the proximal edges 157 of the side flaps 147 may be sandwiched between the side flaps 147 and the interior surface 102 of the chassis 100.

The interior surface of each interior abdominal stretch panel may contact the skin of the wearer when the diaper 20 is worn. In this case, the layer forming the interior surface may be formed of a soft material that will not irritate the skin of the wearer. Many suitable materials are known in the art, including rayon and synthetic nonwovens, such as spunbonded or carded polypropylene, polyethylene, or polyester or other olefinic materials.

An abdominal stretch panel may have a laminate structure. In particular, an abdominal stretch panel may include an interior layer and an additional layer or layers disposed exteriorly of the interior layer. An elastic version of one of the aforementioned suitable materials, may comprise a nonwoven exhibiting substantial elastic properties, such a material may be used for any of the layers. This abdominal stretch panel may include both an interior skin-contacting layer and one or more elastic layers laminated to the interior layer. Suitable materials for the elastic layer are well-known in the art, including natural rubber strands, synthetic rubber strands, elastomeric films, etc. The material chosen for the elastic layer may exhibit a force response proportional to its elongation. The abdominal stretch panel may also include an exterior cover layer laminated to the elastic layer on its surface opposite the interior layer, thereby forming a trilaminate in which the elastic layer is sandwiched between the interior layer and the exterior cover layer.

A combination of lamination methods may be used, if desired, so long as they are suitable for the elements being attached together and their constituent materials. For abdominal stretch panel embodiments comprising an elastic film and a nonwoven, the film and nonwoven may differ in size both laterally and longitudinally. For example, an abdominal stretch panel may comprise a laminate of film and nonwoven wherein the film extends laterally only partially across the width of the nonwoven of the abdominal stretch panel. In yet another embodiment, the film layer of the abdominal stretch panel may extend longitudinally only partially across the longitudinal length of the nonwoven of the abdominal stretch panel. Such designs minimize the amount of active stretch material and help minimize overall cost of the structure.

An abdominal stretch panel may have uniform extension characteristics throughout its area or may have different extension characteristics in different portions. For example, a portion of an abdominal stretch panel may be made laterally extensible to a maximum extensibility greater than a maximum extensibility of an adjacent portion, such that a desired fit on a wearer can be achieved. The difference in extensibility may be provided by varying the lamination and/or activation process, for example by varying the localized elongation in an activation process.

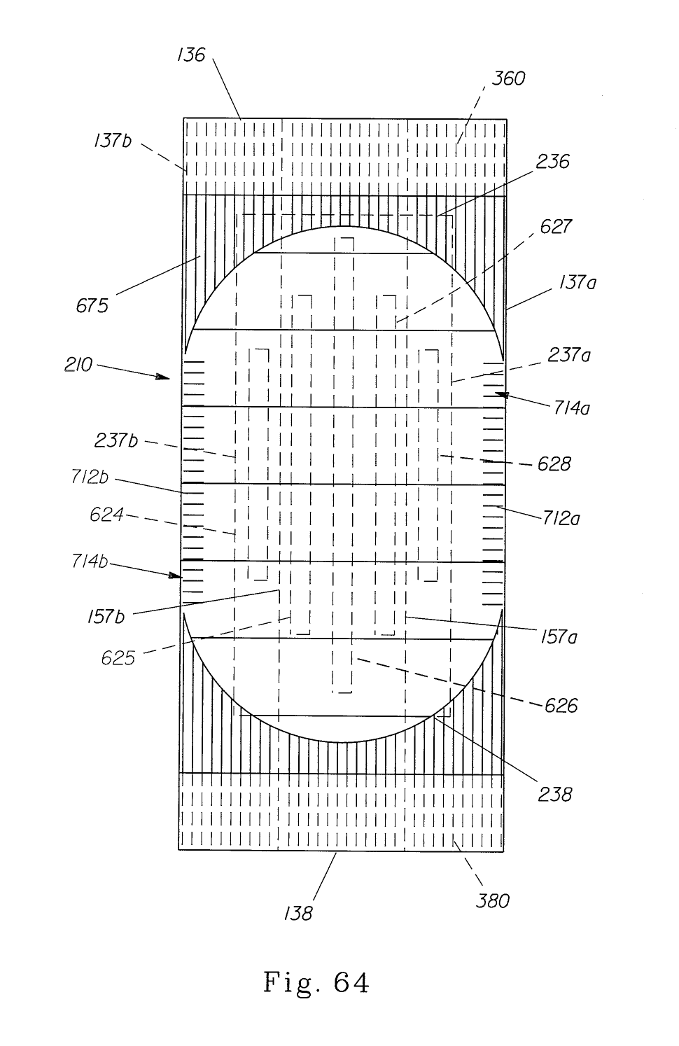

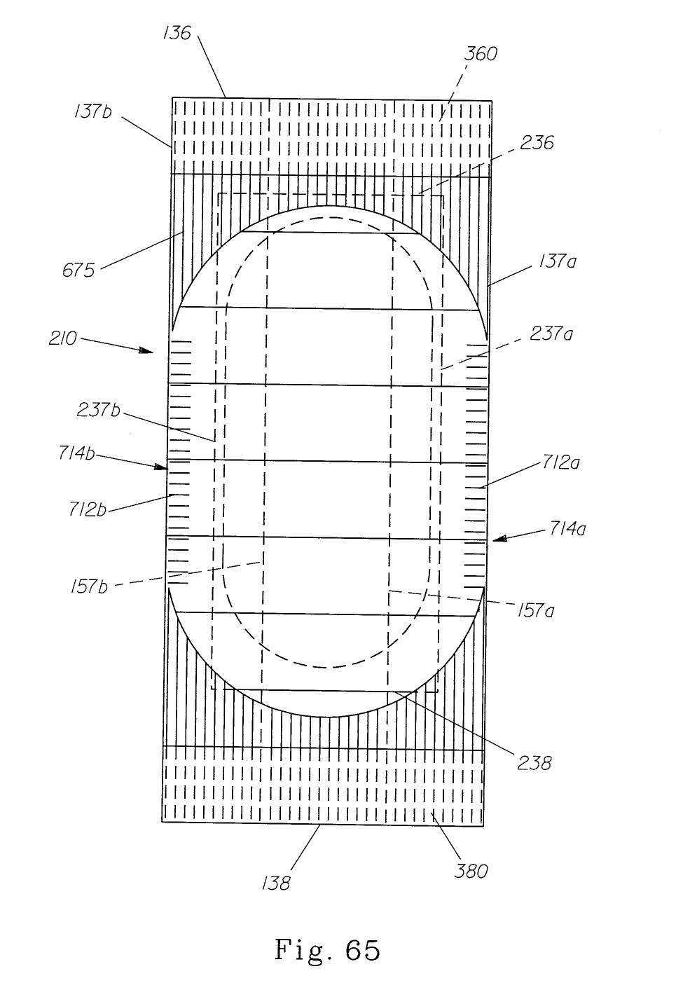

Additionally, as shown in FIGS. 64 and 65, a portion of one or a combination of the backsheet, side barriers, and cuff flaps may be activated as described above to provide a greater level of longitudinal extensibility. In such an embodiment, at least one of the opposing rolls may include a staggered orientation of teeth such that when engaged with the opposing roll, a curved or shaped activation pattern is formed in the web. This curved pattern may be useful for activating the side barriers of a shaped diaper (e.g., a side notched diaper) or useful for activating around particular diaper elements (e.g., graphic elements). Alternatively, the teeth of the rolls may be oriented in a substantially non-staggered pattern thereby creating a longitudinally extending area of extensibility that comprises a substantially straight pattern formed in the web. The depth to which the teeth intermesh, i.e. depth of engagement, will determine to what degree the web is incrementally stretched and/or plastically deformed and therefore the degree of extensibility imparted to the web.

In some embodiments, the chassis web 149 can be activated in a portion of side barriers or cuff flaps. For example, in these embodiments, as shown in FIGS. 64 and 65, the portion of the chassis immediately adjacent the side edges can comprise laterally oriented alternating ridges 712 and valleys 714 while other regions may comprise longitudinally oriented alternating ridges 312 and valleys 314.

In certain embodiments, the activated portion of the chassis web 149 disposed in the front or back waist region may comprise a longitudinally continuously activation pattern (e.g., via ring rolling) and a longitudinally intermittent activation pattern (i.e., a structured elastic-like formed web material). These two activation patterns may be disposed in an overlapping or side-by-side relationship.

In certain embodiments, the activated portion of the chassis web 149 disposed adjacent the side edges, for example, the side barriers and/or cuff flaps may comprise a laterally continuous activation pattern comprising laterally oriented ridges 712 and valleys 714 (e.g., via ring rolling) and a longitudinally intermittent activation pattern (i.e., a structured elastic-like formed web material). These two activation patterns may be disposed in an overlapping or side-by-side relationship. In other words, the overlapping orientation of the two distinct types of activation may be performed sequentially on the same region of the diaper. For example, as a more particular embodiment, the entire chassis web 149 may be longitudinally intermittently activated in the machine direction to form a structured elastic-like formed web material and the portion of the chassis web 149 forming the side barriers and/or cuff flaps may be continuously activated to form laterally oriented ridges 712 and valleys 714, such that the side barriers and/or cuff flaps are activated in both the longitudinal and lateral directions.

In the finished diaper, the chassis may be non-rectangular, but instead have an overall shape in plan view of a “T” or of an “I”. Such a non-rectangular configuration may impart a tailored appearance to the diaper 20 when it is worn and may also impart an impression that the diaper 20 will fit comfortably between the legs of a wearer.

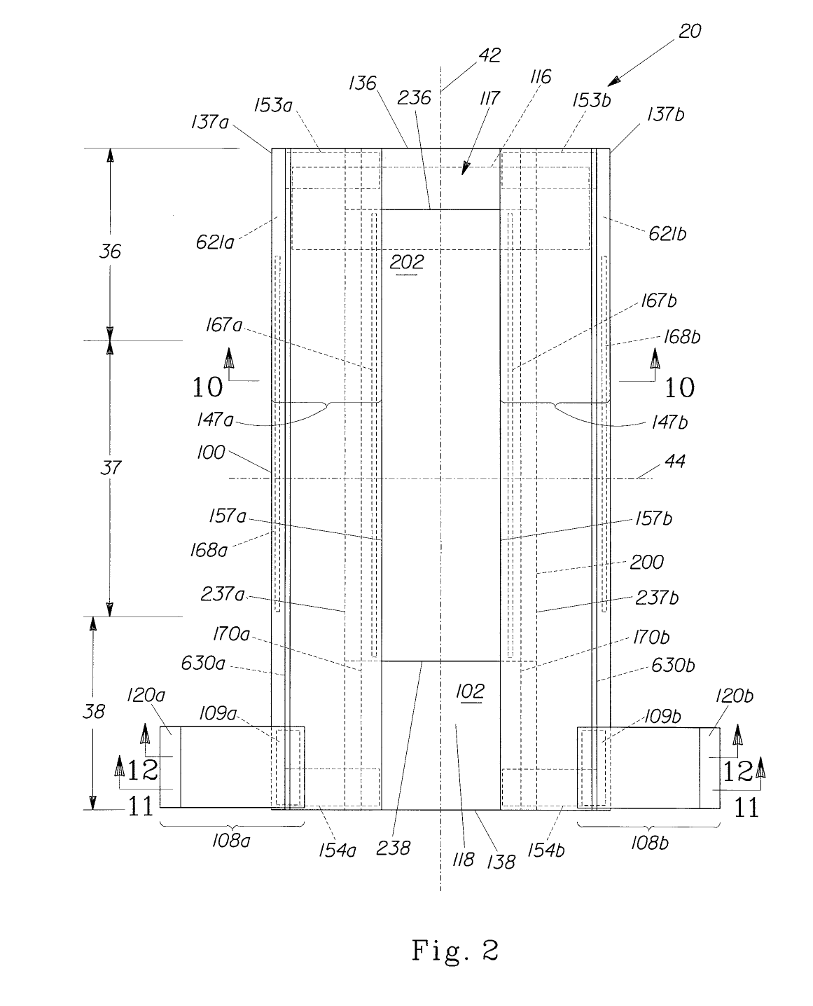

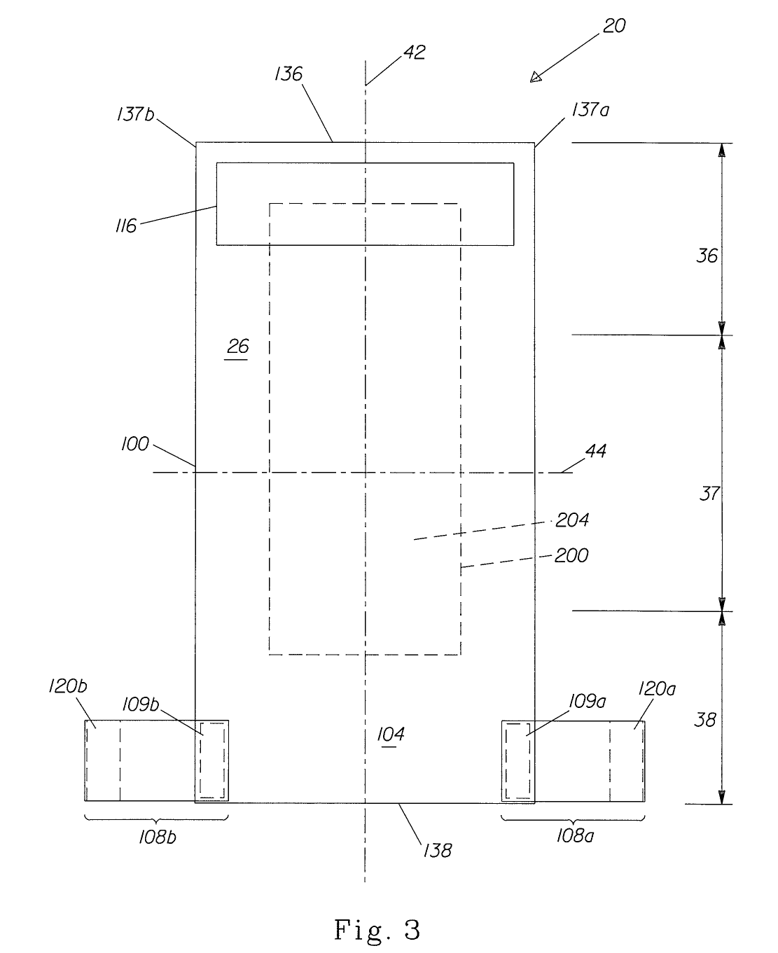

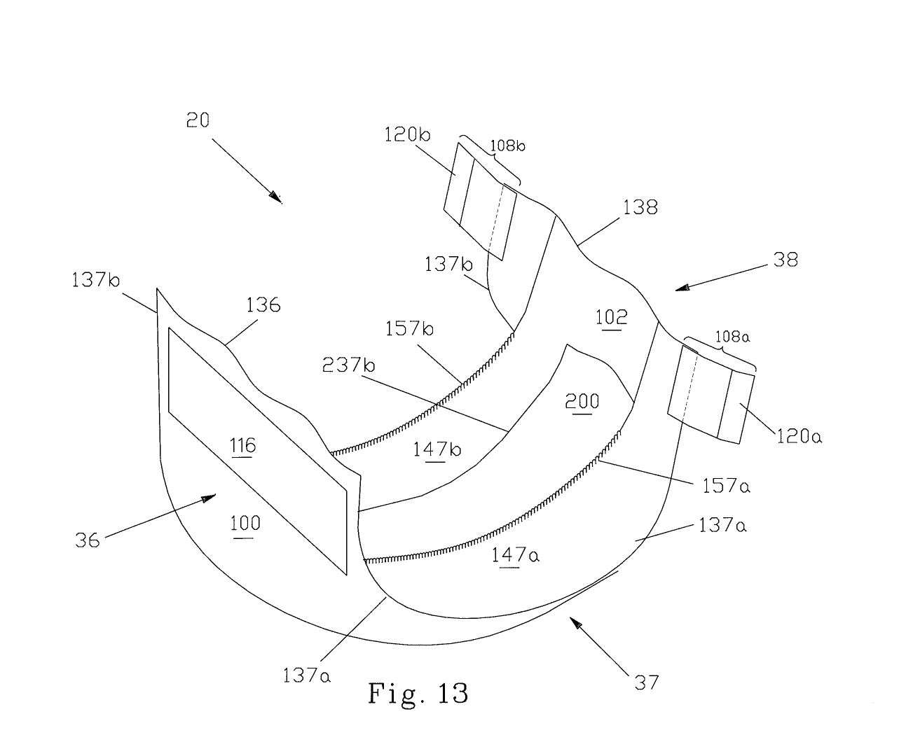

An exemplary non-rectangular configuration of the chassis is shown in FIG. 2, FIG. 3, and FIG. 13. As shown in these figures, laterally opposing belt ears 108 in the back waist region 38 extend laterally outward while the adjacent side flaps 147 remain folded laterally inward. The laterally outwardly extending belt ears 108 impart a “T” shape to the diaper. Alternatively, laterally opposing belt ears disposed in the front waist region 36 may similarly extend laterally outward to impart a “T” shape to the diaper. In some embodiments, both front belt ears and back belt ears may extend laterally outward while the adjacent side flaps 147 remain folded laterally inward, in which configuration an “I” shape is imparted to the diaper 20.

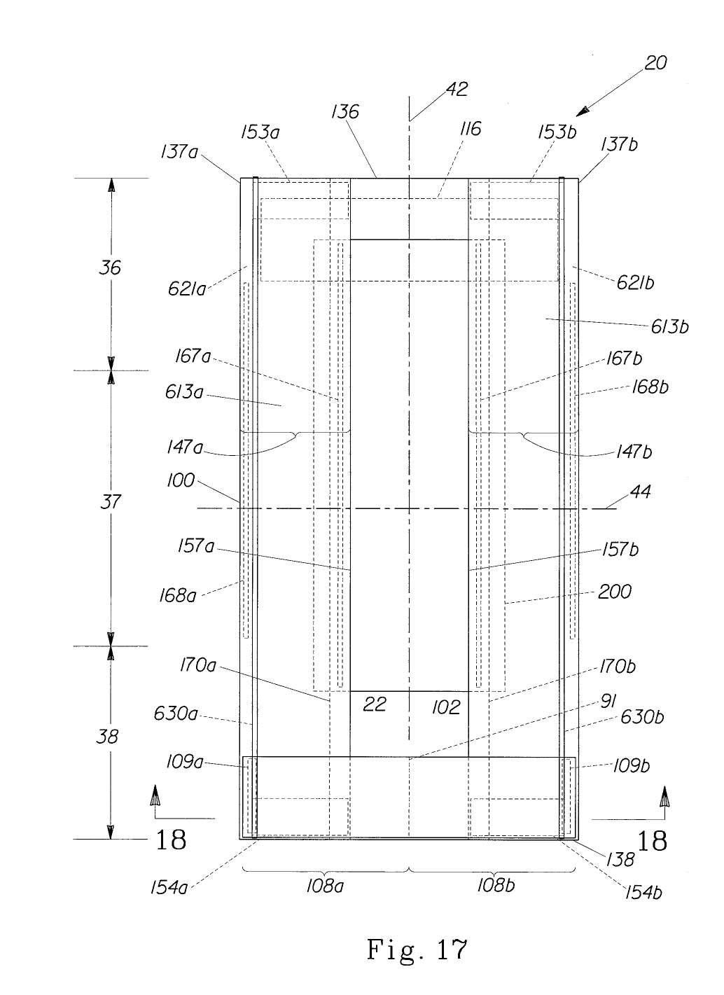

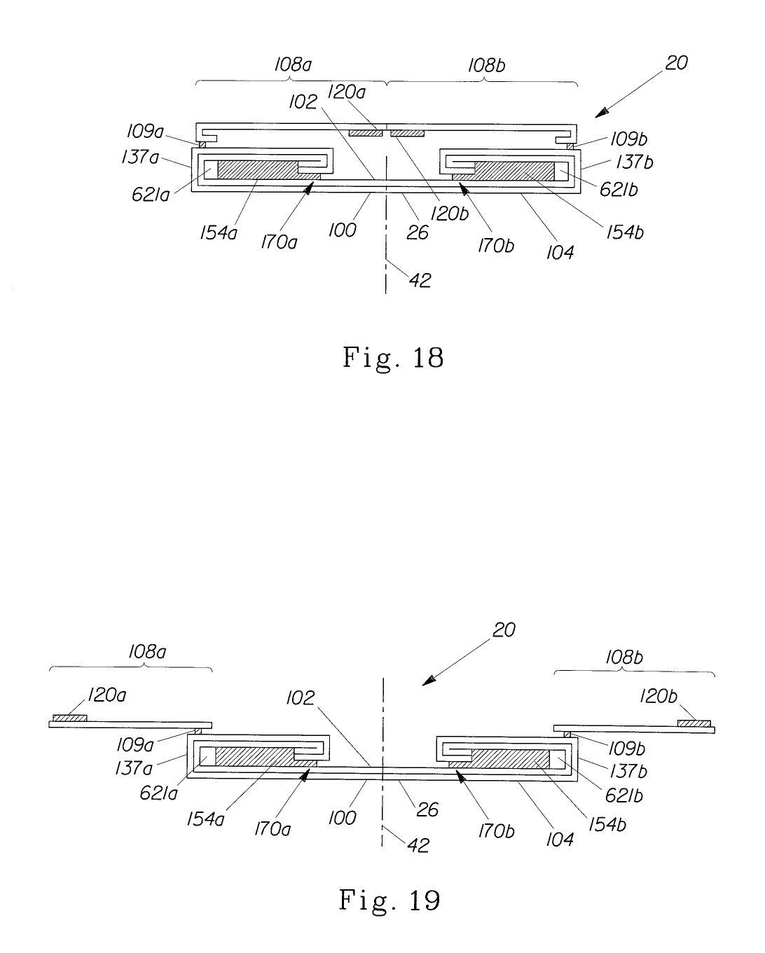

Each belt ear may be attached to chassis at or adjacent the side edges 137 in an ear attachment zone 109. The belt ears may be attached interiorly to the respective side flap 147 in ear attachment zone 109, as shown in FIG. 1, FIG. 2, FIG. 3, FIG. 7, FIG. 9, FIG. 11, and FIG. 12. Alternatively, the belt ears may be attached exteriorly to the backsheet 26 at or adjacent the side edges 137. For ease of manufacturing and packaging, the belt ears 108 may remain disposed laterally inward until a user desires to deploy them for use when applying the diaper 20 onto the body of a wearer. For this purpose, as shown in FIG. 17 and FIG. 18, one edge of each belt ear 108 may be defined by a frangible line of attachment 91 along which the belt ear can be detached from the laterally opposing belt ear for deployment laterally outward in preparation for use as illustrated in FIG. 19. Such a frangible line of attachment 91 may be formed in a layer or a laminate of layers by perforation, by the formation of a brittle area or areas at which the material will preferentially fracture when stressed, by the formation of a weaker area or areas at which the material will preferentially tear when stressed, by the formation of a friable area or areas at which the material will preferentially crumble when stressed and/or bent, or by any other method of providing frangibility that may be suitable for the materials involved.

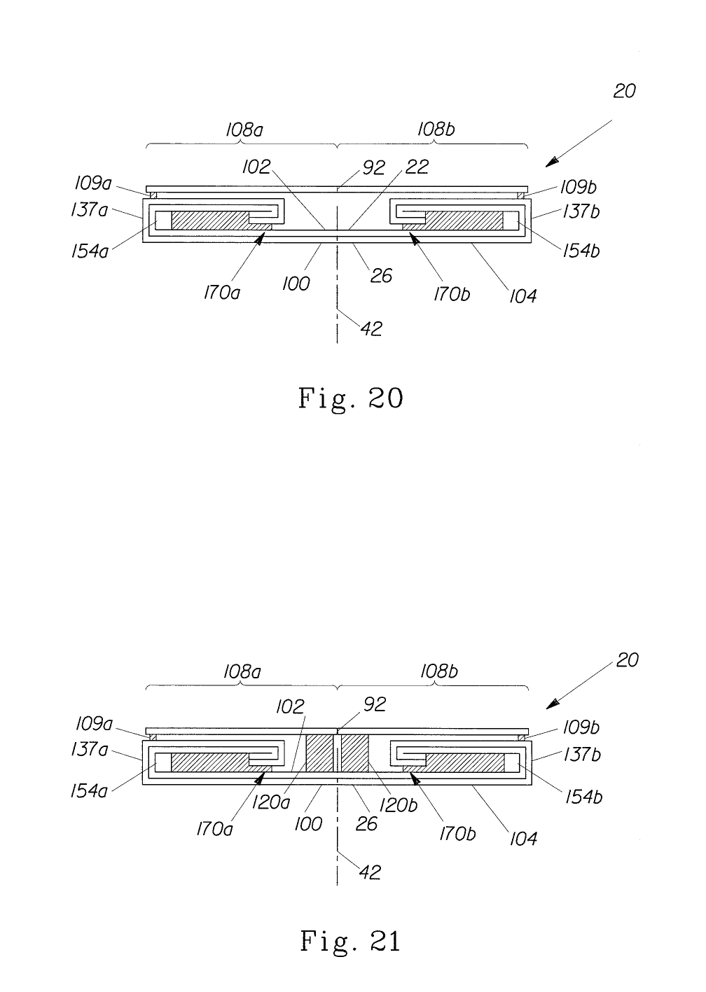

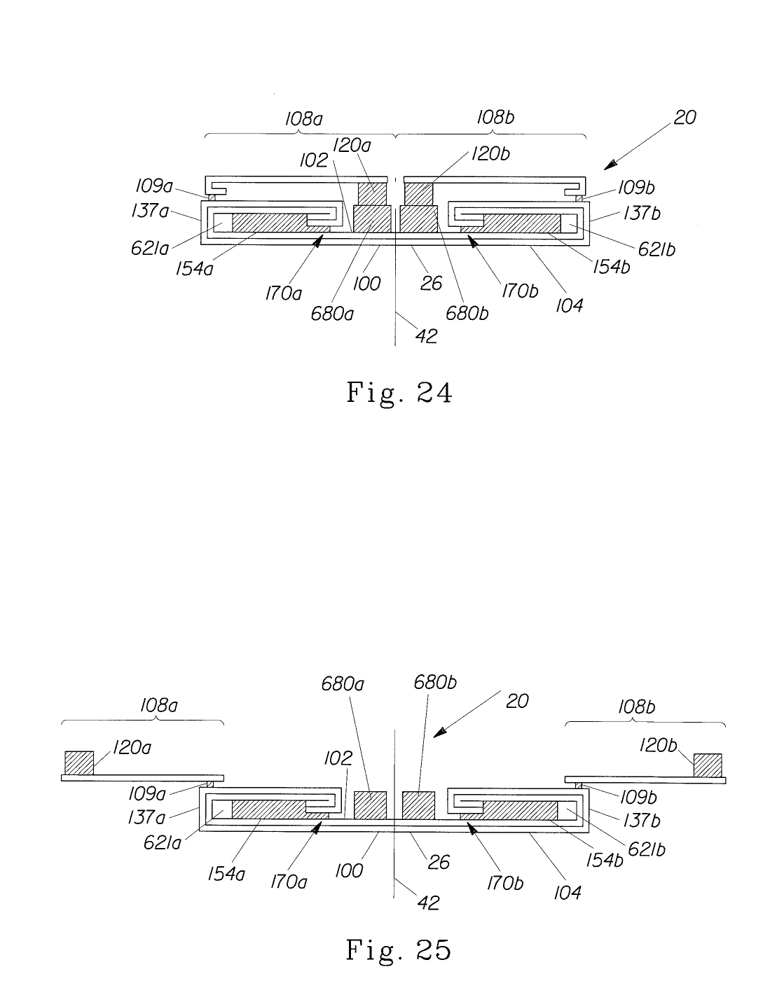

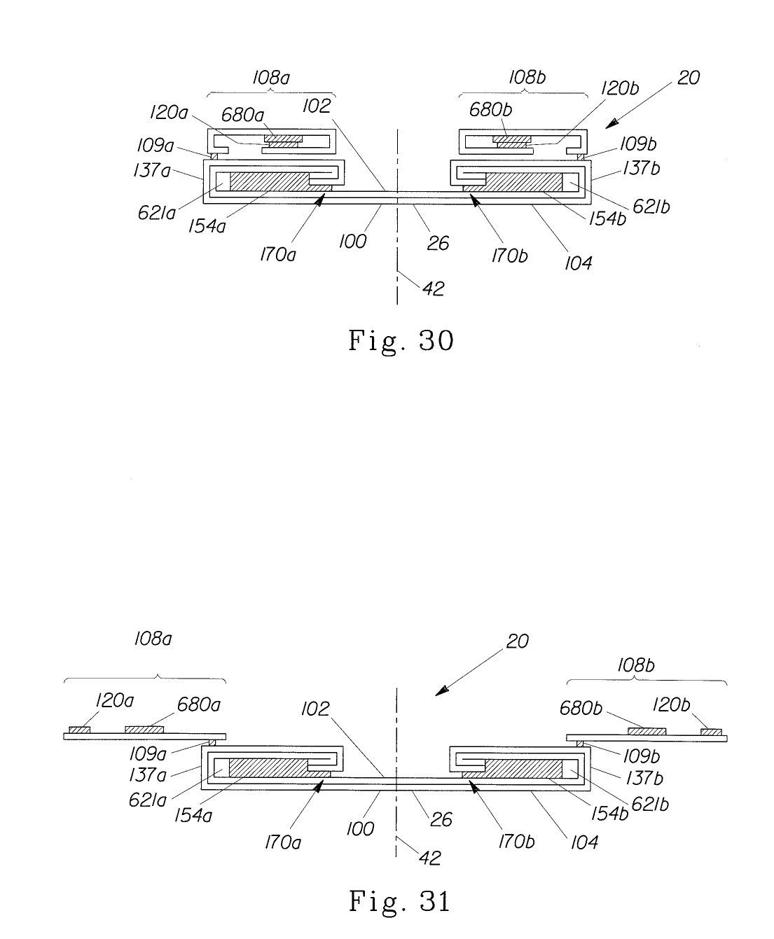

Alternatively, as shown in FIG. 20 and FIG. 21, one edge of each belt ear 108 may be defined by a cut line 92 at which the belt ear is severed from the laterally opposing belt ear during manufacture. Because the formation of this cut line 92 would allow the belt ear 108 to deploy prematurely, the belt ear 108 may be held laterally inwardly disposed by a releasable attachment member until being released and deployed laterally outward so as to project laterally outward beyond the adjacent side flap. As shown in FIG. 21, a fastening element 120 may serve to releasably attach the belt ear 108 to the interior surface 102 until it is deployed for use. Such a releasable attachment may also be used in combination with a belt ear 108 that is defined by a frangible line of attachment 91 if additional assurance is desired that the belt ear 108 will not inadvertently be deployed prematurely, for example by handling that might rupture the frangible line of attachment. Alternatively, as shown in FIGS. 24 and 25, the fastening element 120 may be releasably attached to an attachment surface 680. The attachment surface 680 may be a release tape, mating loops, a lacquered portion of the interior surface 102 of the chassis 100 (as shown in FIGS. 24 and 25), etc. Further, as shown in FIGS. 30 and 31, the fastening element 120 may be releasably attached to the attachment surface 680 on the belt ear 108. In such an embodiment, the belt ear 108 may be folded over itself and positioned over the side flap 147.

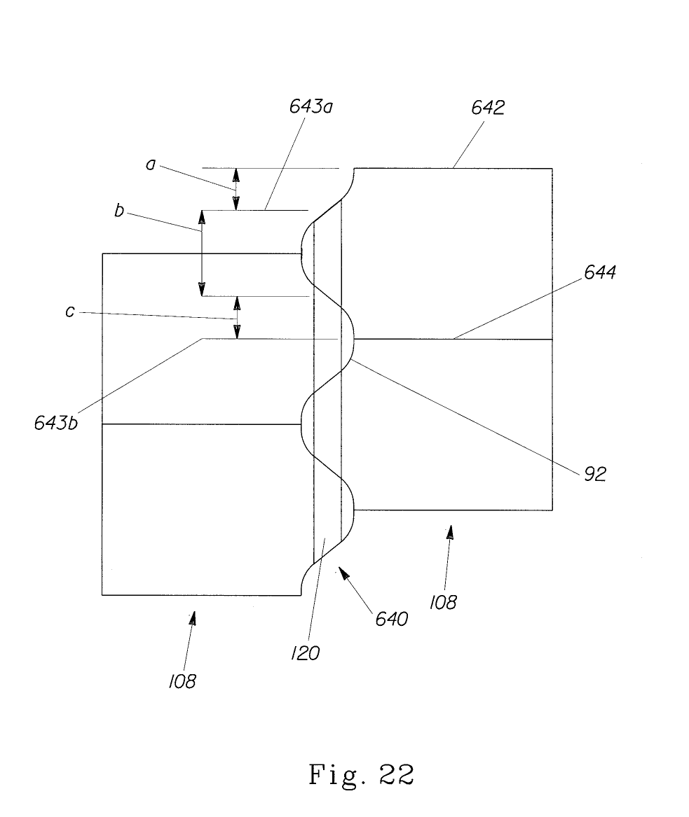

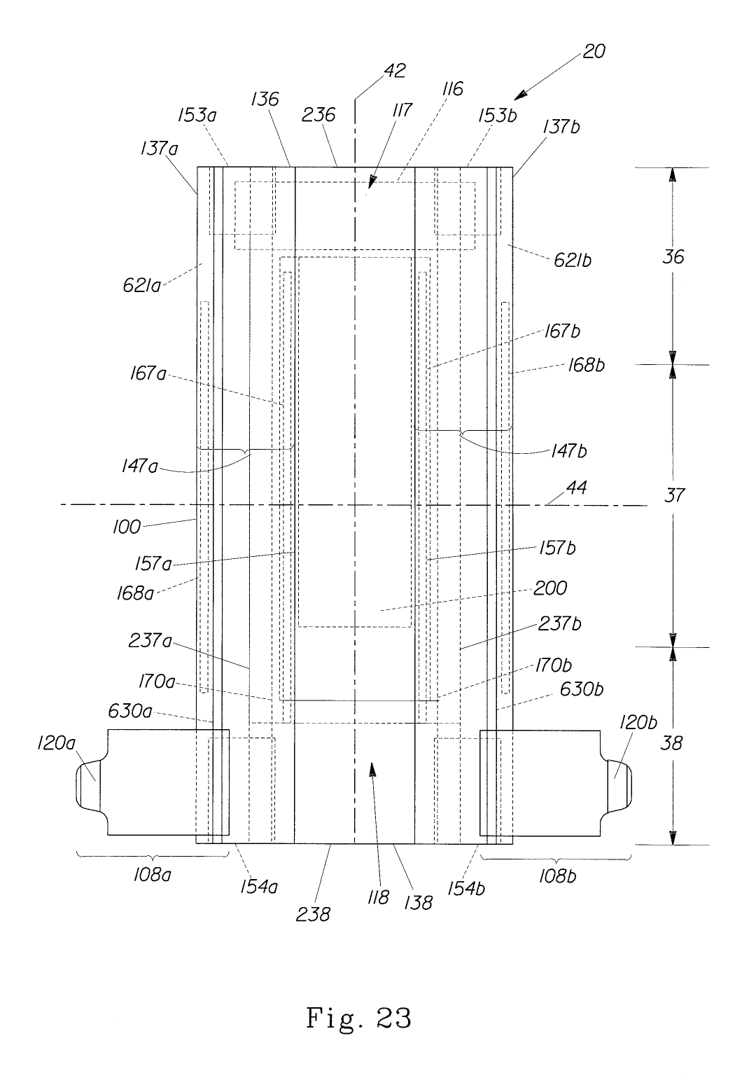

The belt ears 108 may be created from a single web via a nested cut line 92. The following constraints should be maintained in forming the nested configuration of the belt ear 108 as illustrated in FIG. 22. The most important constraint may be the longitudinal distances of a tab portion 640 relative to a remainder 641 of the belt ear 108. With further reference to FIG. 22, the distance b=a+c where a is the distance from the first edge 642 of the belt ear 108, to a midpoint 643a through the transition between first edge 642 of the belt ear 108 and a midpoint of the tab portion 640 of the belt ear 108, the distance c is from the midpoint 643b of the transition between the midpoint of the tab portion 640 and the second edge 644 of the belt ear 108. The distance b is from the midway point 643a to midpoint 643b. When the fastening element 120 is applied to the belt ear prior to cutting the web, the nested pattern cut line 92 also cuts through and shapes the fastening element 120 to match the portion of the tab 640 underlying the fastening element 120.

In an embodiment of the invention (not shown), a single width web of belt ear material such as a nonwoven material may be fed from a roll into a die cutter to provide one main web cut line 92 having the desired pattern of the final belt ear. In another embodiment, the two now separate webs are phased and aligned, with the tab portions (e.g., 640) positioned in a partially overlapping configuration. The combined web may then be cut to length and the pairs of belt ears are placed onto the chassis (e.g., 100) and attached as described herein.

In a further embodiment (not shown), the two separate webs are in the same plane. In another embodiment, after the tab portions (e.g., 640) of the webs are aligned and placed in a “gap” format, i.e., the webs are not touching, the webs are cut into belt ear pieces and placed onto the chassis (e.g., 100) and attached as described herein. The belt ears may be held in a closed configuration by fasteners or other means also described further herein.

Portions of the diaper 20 can be fastened together to encircle the waist and the legs of the wearer in many well-known ways. For example, separate fastening devices such as safety pins, separate tapes, a separate tie strap or straps, and/or a separate belt can be used for this purpose. Alternatively or in addition, fastening elements can be incorporated into the diaper 20 to enable a user to apply the diaper to the body of the wearer without, or in conjunction with, any separate fastening devices. Many suitable types of such incorporated fastening elements are well-known, including, for example, tapes, adhesives, adhesive tape tabs, ties, buttons, hooks, loops, snap fasteners, other forms of mechanical fasteners, cohesive patches, etc. When configured for use, portions of these incorporated fastening elements may project laterally or longitudinally outward or they may lie entirely inside the edges of the diaper 20 (consistent with the disclosure herein, the edges of the diaper 20 encompass the belt ears).

For example, laterally opposing fastening elements 120 may be attached to the belt ears. These fastening elements 120 may be disposed on the back belt ears 108, as shown in FIG. 1, FIG. 2, FIG. 3, FIG. 7, FIG. 9, FIG. 11, FIG. 12 and FIG. 13. Such fastening elements 120 may be used to fasten the back belt ears 108 to the front waist region 36 or to fasten the back belt ears 108 to front belt ears if present. Fastening elements may similarly be disposed on front belt ears and may be used to fasten the front belt ears to the back waist region 38 or to fasten the front belt ears to back belt ears 108, if present. In some embodiments in which both front belt ears and back belt ears are present, fastening elements may be attached to one but not the other. In other embodiments in which both front belt ears and back belt ears are present, complementary fastening elements may be attached to the respective belt ears such that the front belt ears may be fastened to their corresponding back belt ears. Fastening elements may be disposed on a waist region not having belt ears extending from it and may be used to attach that waist region to belt ears extending from the opposing waist region. Fastening elements 120 may be disposed on both the back belt ears 108 and the front belt ears. Such fastening elements 120 may be used to fasten the respective left and right pairs of such ears together or to fasten the respective belt ears to the opposing waist regions.

Optionally, a fastening sheet 116 may be attached onto the exterior surface 104 of the chassis 100, as described in U.S. Patent Application Publication No. 2005/0171499A1 to Nigam et al. When a fastening sheet 116 is provided, adhesive fastening elements 120 may be adhered to the fastening sheet 116 to fasten the back waist region 38 and the front waist region 36 together, or mechanical fastening elements 120 (e.g., hooks) may engage with the fastening sheet 116 (e.g., loops) for the same purpose. The incorporation of such a fastening sheet 116 may be desirable, for example, in order to make it possible to use a relatively inexpensive and relatively weak material for the backsheet 26.

Several configurations of cohesive fastening patches are described in U.S. Patent Application Publication No. 2005/0171499A1 to Nigam et al. In the present invention, cohesive fastening patches may be disposed on the belt ears. For example, in FIGS. 1, 2, 3, and 13, the back fastening elements 120 may be formed by cohesive fastening patches and the complementary front fastening sheet 116 in the front waist region 36 may be formed of a compatible cohesive fastening material.

Alternatively, when a laminate backsheet is used and is oriented with the nonwoven disposed exteriorly, some forms of mechanical fasteners that typically require specific mating fastener elements, such as hooks that typically mate with loops, may be configured to engage with the nonwoven and thereby make the inclusion of the specific mating fastener element or fastening sheet unnecessary. For example, the fastening elements 120 may be formed by hook fastening patches configured to engage with the nonwoven layer 31 of the laminate backsheet 26. A laminate backsheet 26, such as a laminate of a film 30 and a nonwoven 31, may also be suitable for use with the nonwoven 31 disposed interiorly to separate the film 30 from the skin of the wearer, or with nonwovens 31 disposed both exteriorly and interiorly. In addition, the nonwoven 31 and film 30 layers of the multi-layer laminate backsheet 26 may have different widths, for example the film 30 may be wider than the nonwoven 31.

In the exemplary embodiments shown in cross section in FIG. 20, and FIG. 21, each belt ear 108 lies flat while laterally inwardly disposed and may be folded adjacent to ear attachment zone 109 where it may be attached to the respective side flap 147 in order to be deployed laterally outward for use. In this configuration, the ear attachment zone 109 is predominately subjected to a peel force when the belt ear 108 is subjected to a tensile force as during normal wear of the diaper 20. Therefore, it may be desirable to attach the belt ear 108 by a through-bonding method, i.e., a bonding method that fuses the layers of material together, such as pressure bonding or thermal bonding, in order to maximize the peel strength.

Another exemplary attachment configuration is shown in FIGS. 1, 7, 9, 18 and 24, in which each belt ear is folded adjacent to its ear attachment zone 109 where it is attached to the respective side flap 147 while laterally inwardly disposed and is unfolded, i.e., made to lie flat, in order to be deployed laterally outward for use. In this configuration, the ear attachment zone 109 is predominately subjected to a shear force when the belt ear is subjected to a tensile force. Because the shear strength of an adhesive bond is often greater than its peel strength, an adhesive bond may be used to form the ear attachment zone 109 in this configuration, so long as suitable materials are used.

The ear attachment zones 109 are disposed at or adjacent the side edges 137 in one or both or the waist regions 36 and 38 of the chassis 100. The ear attachment zone 109 may be disposed completely in the area between the side edges 137 and the side barrier attachment zone 630 as illustrated in FIGS. 18, 19, 20, 21, 24 and 25. In such an embodiment the strength of the attachment can be impacted by the individual layers (e.g., film 30 and nonwoven 31 layers (when present)) forming the channels 621 since the individual layers may strain and tear independently when the belt ear 108 is subjected to a tensile force as during normal wear of the diaper 20. In order to improve the strength of the attachment between the belt ears 108 and the chassis 100 it has been determined that if the ear attachment zones 109 extend laterally inward to or beyond the side barrier attachment zone 630 as illustrated in FIGS. 1, 2, 7, 9, 11 and 12, the layer (e.g., film 30) forming the side flap 147 and the layer or layers (e.g., film 30 and nonwoven 31 layers) forming the outer surface 104 of the chassis 100 act together in concert to provide a more robust base structure for attachment of the belt ear 108.

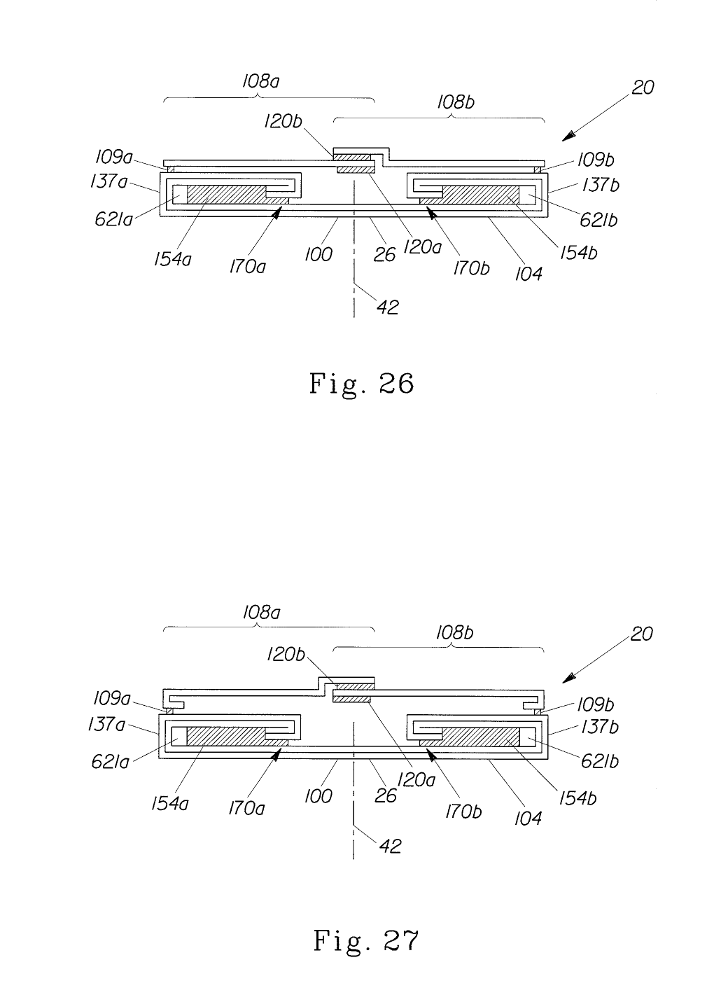

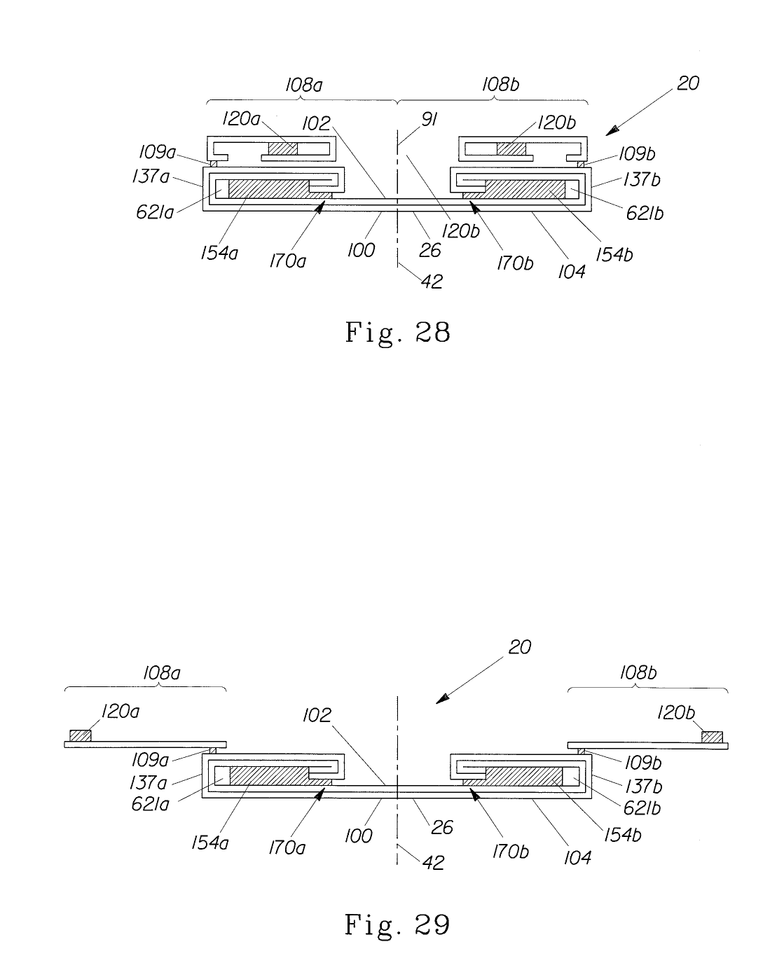

In the exemplary embodiments shown in cross section in FIG. 7, FIG. 9, FIG. 18, FIG. 20, FIG. 21 and FIG. 24, the belt ears 108 do not overlap each other while laterally inwardly disposed. Other exemplary configurations are shown in FIG. 26 and FIG. 27, in which the belt ears 108 overlap while in this disposition. As previously mentioned, for ease of manufacturing and packaging, the belt ears 108 may remain disposed laterally inward until a user desires to deploy them for use when applying the diaper 20 onto the body of a wearer. Therefore, overlapped belt ears may be releasably attached to each other where they overlap. For example, in each of FIG. 26 and FIG. 27, a fastening element 120 serves this purpose.

The belt ears 108 may be folded such that they form a double layer belt ear 108 while disposed laterally inward prior to deployment as illustrated in FIGS. 28, 29, 30 and 31. The fastening element 120 may be designed to engage with the surface of the belt ear 108 to maintain the belt ear 108 in folded configuration. The folded belt ears 108 can be held laterally inward until deployed for use by suitable means, for example, releasable adhesives, hook/loop attachments, frangible bonds, etc. This assists with the manufacturing of the diaper 20, keeping the belt ears 108 from deploying prematurely.

The interior surface of each belt ear contacts the skin of the wearer when the diaper 20 is worn. Therefore, the layer forming the interior surface may be formed of a soft material that will not irritate the skin of the wearer. Many suitable materials are known in the art, including rayon and synthetic nonwovens, such as spunbonded or carded polypropylene, polyethylene, or polyester or other olefinic materials.

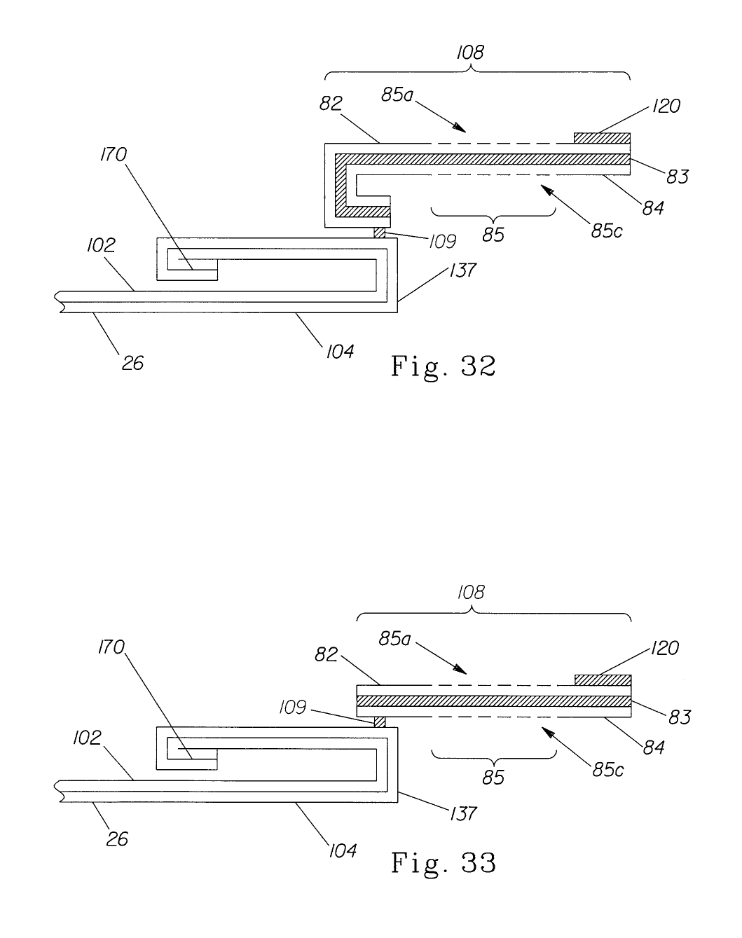

A belt ear 108 may have a laminate structure. In particular, a belt ear 108 may include an interior layer and an additional layer or layers disposed exteriorly of the interior layer. An elastic version of one of the aforementioned suitable materials, such as a nonwoven exhibiting substantial elastic properties, may be used for any of the layers. For example, belt ears 108 having laminate structures are shown in FIG. 32 and FIG. 33. Each of these belt ears includes an interior skin-contacting layer 82 and an elastic layer 83 laminated to the interior layer 82. Suitable materials for the elastic layer 83 are well-known in the art, including natural rubber strands, synthetic rubber strands, elastomeric films, etc. The material chosen for the elastic layer 83 may exhibit a force response proportional to its elongation. Each belt ear 108 may also include an exterior cover layer 84 laminated to the elastic layer 83 on its surface opposite the interior layer 82, thereby forming a trilaminate in which the elastic layer 83 may be sandwiched between the interior layer 82 and the exterior cover layer 84.

The layers of each belt ear may be laminated by any method suitable for the elements being attached together and their constituent materials. For example, the elastic layer 83 may be maintained in a stretched condition while being attached to a relaxed skin-contacting layer 82 (and a relaxed exterior cover layer 84 if present) and then allowed to relax. The resultant contraction of the elastic layer 83 may gather the skin-contacting layer 82 in such a way as to create rugosities and the laminate thus formed may be extended in the direction of the original stretch up to the original dimension of the skin-contacting layer 82 (and the exterior cover layer 84 if present) with only the elastic layer 83 resisting the extension. A similar result may be achieved by, for example, first gathering the skin-contacting layer 82 (and the exterior cover layer 84 if present), such as by pleating it, and then attaching the elastic layer 83 in a relaxed condition. The resultant laminate may be extended in a direction perpendicular to the pleat ridges up to the original dimension of the skin-contacting layer 82 (and the exterior cover layer 84 if present) with only the elastic layer 83 resisting the extension.

In some exemplary methods, the lamination may be performed with both the elastic layer 83 and the skin-contacting layer 82 (and the exterior cover layer 84 if present) relaxed. All or a portion of the resultant laminate belt ear may subsequently be “activated” by subjecting it to elongation to create localized areas of deformation or ruptures in a portion 85a of the skin-contacting layer 82 (and a portion 85c of the exterior cover layer 84 if present). In FIG. 16 and FIG. 17, belt ears having activation portions 85 are shown, with the deformed or ruptured portion 85a of the interior layer 82 and the deformed or ruptured portion 85c of the exterior cover layer 84 shown in dashed lines representing exemplary breaks in and/or separation of the fibers in nonwoven materials. The deformed or ruptured portion 85a of the interior layer 82 (and the deformed or ruptured portion 85c of the exterior cover layer 84 if present) in the resultant activated portion 85 of the laminate provides little or no resistance to extension in the direction of the original elongation. For example, when a nonwoven is used for the interior layer 82 (and the exterior cover layer 84 if present), the deformed or ruptured portion(s) typically include(s) breaks in and/or separation of the fibers that render the ruptured portion(s) substantially incapable of transmitting tensile forces in the plane of the nonwoven. Alternatively, when an extensible nonwoven is used for the interior layer 82 (and the exterior cover layer 84 if present), the deformed portion(s) typically include(s) areas of fiber deformation that render the deformed portion(s) substantially incapable of transmitting tensile forces in the plane of the extensible nonwoven until a substantial level of extension is achieved. Some suitable activation methods are known in the art as “ring-rolling” processes.

A combination of lamination methods may be used, if desired, so long as they are suitable for the elements being attached together and their constituent materials.

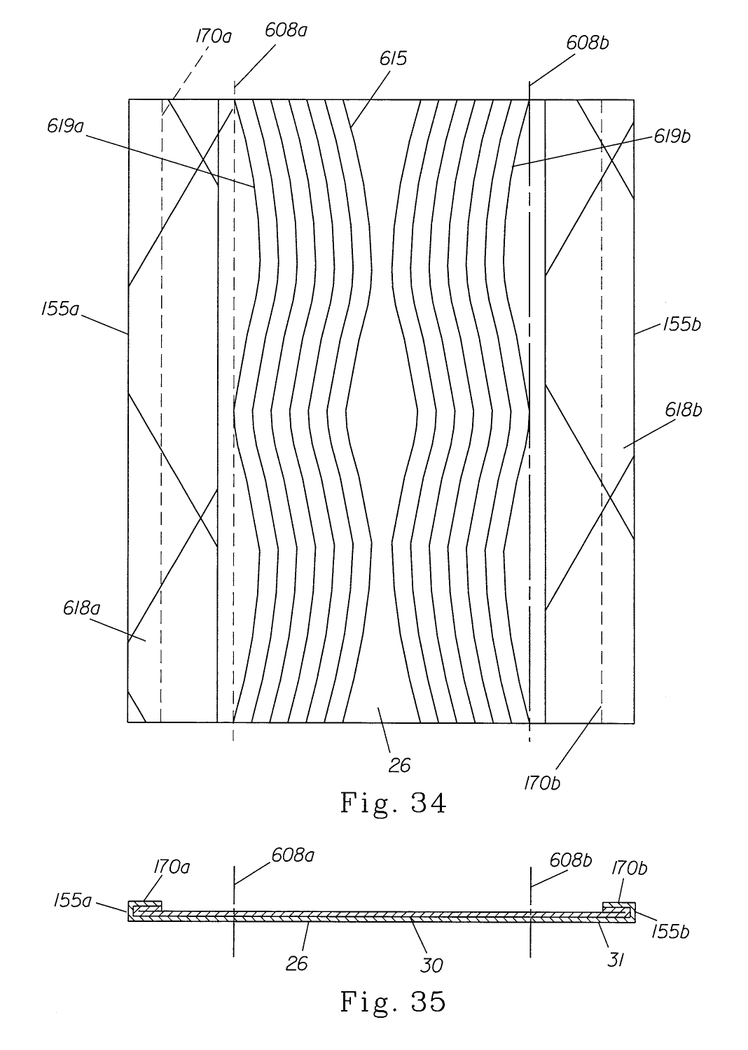

As illustrated in FIGS. 34, 35, 36, 37, 38 and 39, one or more of the layers (e.g., the film 30 and nonwoven 31) of the backsheet 26 may comprise one or more design fields 615 and 618. A first design field 615 may be in contrast to other design fields (e.g., a second design field 618) or to other portions of backsheet 26 or other portions of the viewable surfaces of the diaper 20. The design fields 615 and 618 may comprise one or a combination of individual graphic elements, patterns of graphic elements, and solid or interrupted colored fields. For example, the design fields 615 and 618 may be comprised of a continuously repeating pattern formed in a longitudinally extending direction. Alternatively, the design fields 615 and 618 may be comprised of graphic elements (not shown) designed for a specific size diaper 20 or to provide specific design language associated with a specific region (e.g., front waist region 36, back waist region 38, or crotch region 37 of the diaper 20. Such graphic elements are often referred to as pitched graphics meaning the design is linked or associated with the pitch (i.e., length) of the diaper 20 or portions of the diaper 20 linked to the diaper's pitch.

In certain embodiments of the present invention, particularly those embodiments where a portion of the backsheet 26 is folded to form longitudinally extending laterally opposing side flaps 147a and b, the design fields 615 may extend to cover (or be viewable over) the entirety of the exterior surface 104 of the unfolded portion of the backsheet 26, as well as cover (or be viewable on) the upper surfaces 613a and b of the side flaps 147a and b. To be clear, the design field 615 may be viewable through the nonwoven 31 that forms the exterior surface 104 of the backsheet 26 or the upper surfaces 613a and b of the side flaps 147a and b when printed on the film 30 that the nonwoven 31 may be exteriorly bonded to. In such a case, the design fields 615 and 619 would be viewable due to the properties of the nonwoven 31 (e.g., thinness, transparency, opacity, etc.).

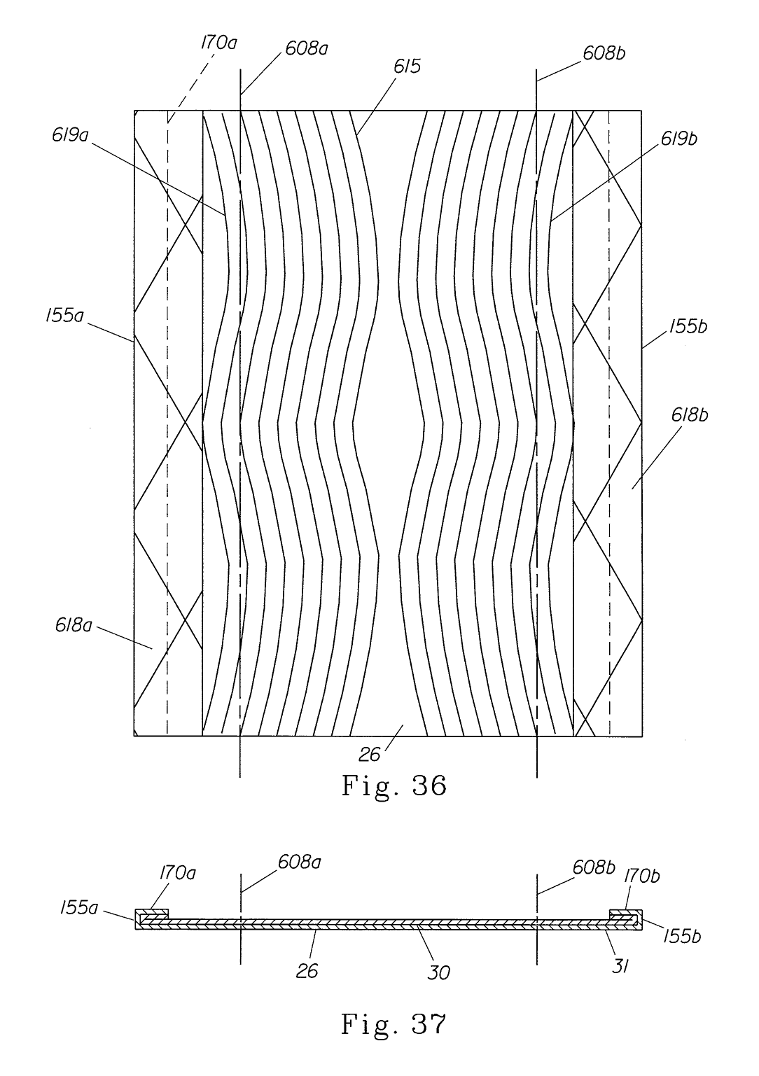

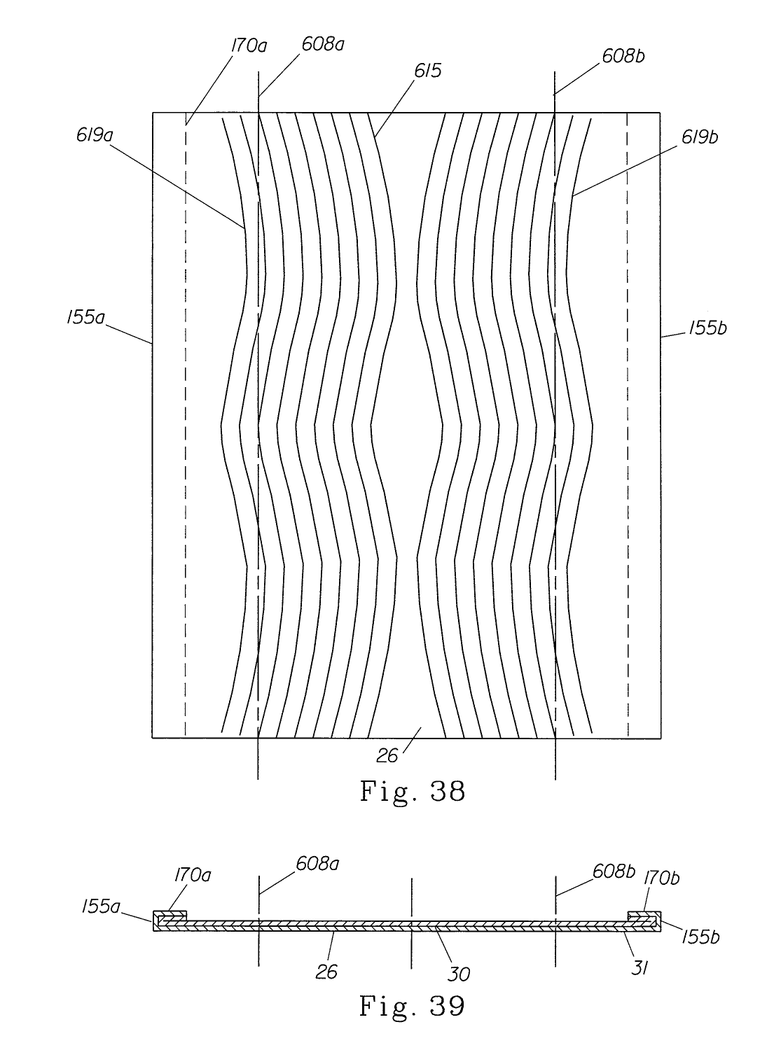

In one embodiment, a first design field 615 extends laterally from one side edge 137a to the laterally opposing side edge 137b of the chassis 100. In addition to covering the exterior surface 104 of the chassis 100 it may be desired to have the first design field 615 wrap over the side edge(s) 137a and b of the chassis 100 and extend laterally inward to cover at least a portion of the side flaps 147a and b. Alternatively, the side flaps 147a and b may comprise a second design field 618. In certain embodiments, the design fields 615 and 618 may provide not only a pleasing aesthetic visual appearance to the exterior of the product, but it may also distinguish the side flaps 147a and b from the unfolded portion of the backsheet 26, or portions of the side flaps 147a and b from itself such that the wearer or caregiver is assisted with proper placement of the side flaps 147a and b during application of the diaper 20. See FIGS. 34 and 35, for example, the backsheet 26 has a first design field 615 undulating along a left fold line 608a of the backsheet 26 and terminating at a right fold line 608b of the backsheet 26. The fold lines 608a and b are the lines that the backsheet 26 is folded along to form side flaps 147a and b and side edges 137a and b. A second design field 618 may extend from the right side edge 619 of the first design field 615 to the unfolded right side edge 155b (which becomes the proximal edge 157b of the side flap 147b when the chassis 100 is folded to form the side flaps 147a and b). Alternatively, as shown in FIGS. 36, 37, 38, and 39, the first design field 615 may extend from a point at or adjacent the left side edge 137a to a point at or adjacent 137b of the chassis 100 such that the side flaps 147a and b become visually distinct elements. Both the first design field 615 and the second design field 618, when present, can make the side flaps 147a and b more visually distinct, especially when a second elastic strand 168a and b is used, such that the double cuff feature (i.e., incorporation of a first and second elastic strands 167a and b and 168a and b, wherein the first elastic strands 167a and b is immediately adjacent the proximal edges 157a and b of the side flaps 147a and b and the second elastic strand 168a and b is oriented immediately adjacent the side edge 137a and b of the chassis 100) is visually highlighted. As illustrated in FIGS. 38 and 39, it should be understood that the double cuff feature may also be highlighted by the first design field 615 being contrasted against side flaps 147a and b which do not comprise a second design field 618, having only the color and texture of the backsheet 26 making it up.

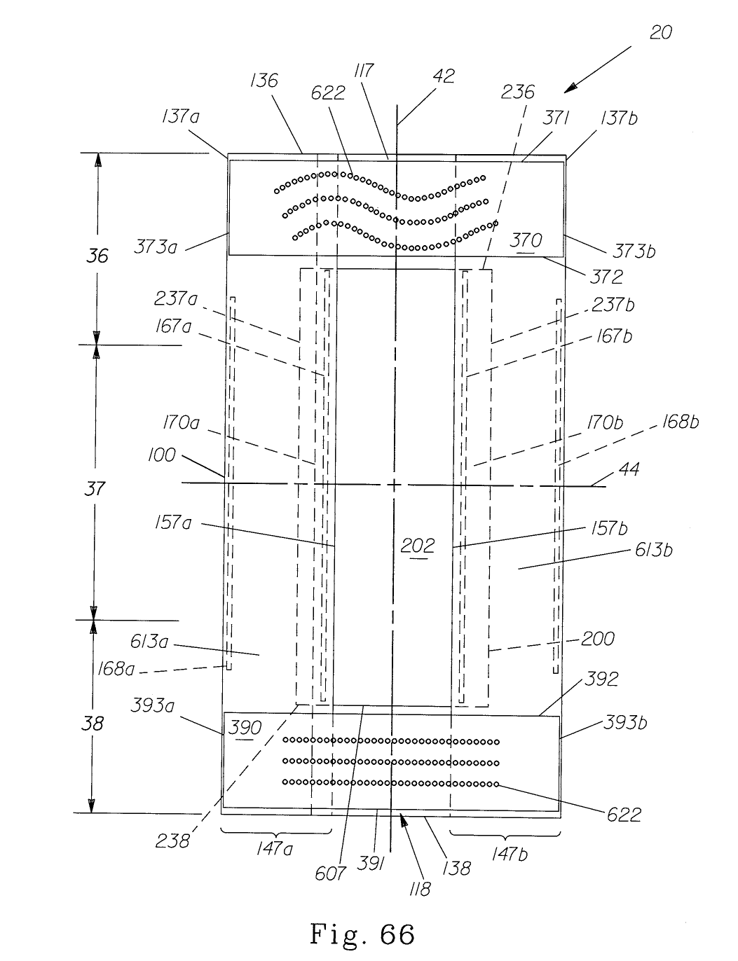

The diaper 20 may comprise apertures 622 as illustrated in FIG. 66 disposed through one or both of the front and back waist regions 36 and 38. For example, the apertures 622 may extend through the back interior abdominal stretch panel 390, the backsheet 26, including the nonwoven 31, and the back exterior abdominal stretch panel 380 to provide air passages for breathability. The apertures 622 may be formed by any suitable means known in the art including hot needle aperturing, laser cutting, die cutting, slitting, etc. One such method achieves apertures 622 using an apparatus (not shown) comprising a pair of counter-rotating, intermeshing rollers, wherein a first roller comprises circumferentially-extending ridges and grooves, and a second roller comprises teeth being tapered from a base and a tip, the teeth being joined to the second roller at the base, the base of the tooth having a cross-sectional length dimension greater than a cross-sectional width dimension; and moving the back interior abdominal stretch panel 390, the backsheet 26, (which may comprise a film layer 30, a nonwoven 31 or both), and the back exterior abdominal stretch panel 380 through a nip of the counter-rotating, intermeshing rollers; wherein apertures 622 are formed in the back interior abdominal stretch panel 390, the backsheet 26, (which may comprise a film layer 30, a nonwoven 31 or both), and the back exterior abdominal stretch panel 380 as the teeth on one of the rollers intermesh with grooves on the other of the rollers. An exemplary method of forming the apertures 622 is disclosed in U.S. application Ser. No. 11/249,618 to O'Donnell (U.S. Pub. No. 2006-0087053).

The number of apertures 622 per unit area of apertured web (i.e., the area density of apertures 622) can be varied from about 1 aperture 622 per square centimeter to as high as 60 apertures 622 per square centimeter. There can be at least 10 or at least 20 apertures 622 per square centimeter, depending on the end use. In general, the area density need not be uniform across the entire area of web, but apertures 622 may be oriented in only certain regions (e.g., the front or back waist regions 36 and 38) of the chassis 100, and can be disposed in a variety of shapes, including lines, stripes, bands, circles, and the like.

The abdominal stretch panels attached to the chassis as described herein are desirable from the standpoints of comfort and appearance. For example, unlike typical stretch waistbands, each abdominal stretch panel covers some portion of a waist region of the diaper 20, i.e., is disposed on one or both of the interior and exterior surfaces rather than being hidden between layers of the chassis 100. Therefore, if the abdominal stretch panel is formed from soft and attractive materials, such as one of the aforementioned nonwovens, the exposed abdominal stretch panel can provide a finished appearance resembling that of cloth underwear and thereby convey an impression of softness and comfort to the user.

### Absorbent Assembly

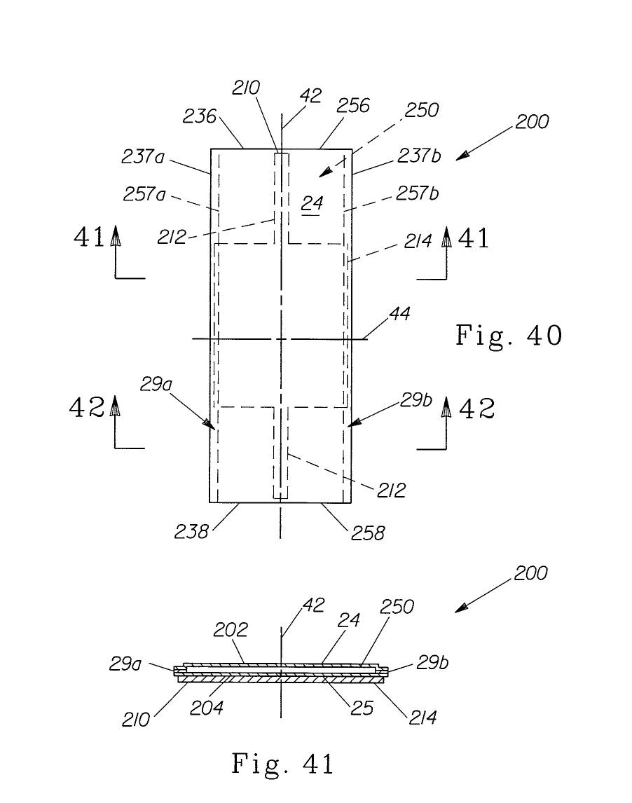

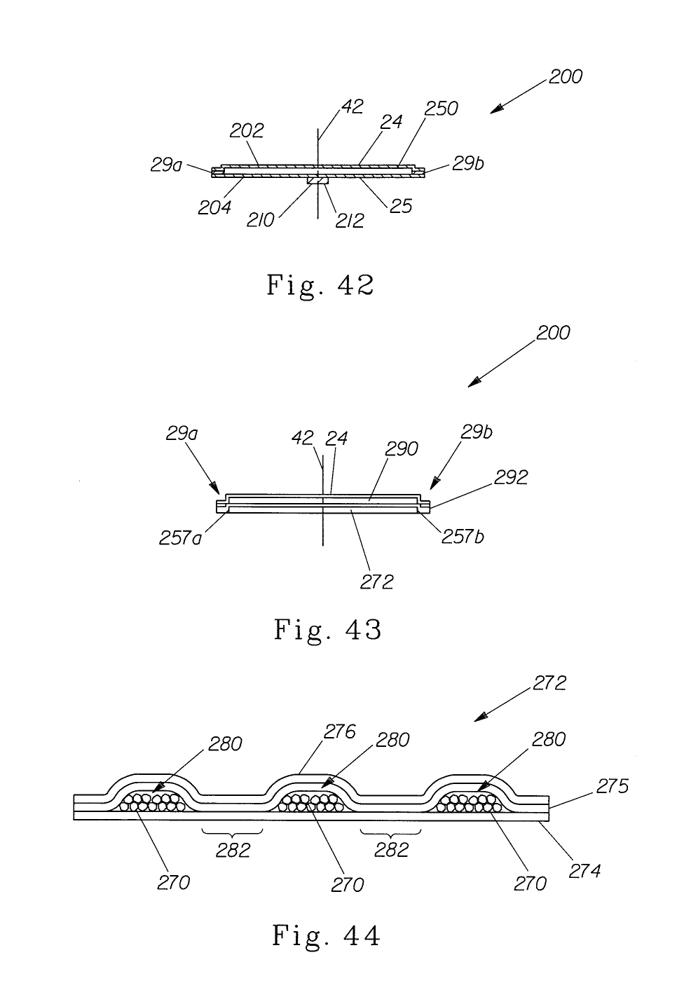

As shown in FIG. 40, FIG. 41, FIG. 42, FIG. 43 and FIG. 44, the absorbent assembly 200 includes an absorbent core 250. The absorbent core 250 has a laterally extending front edge 256, a longitudinally opposing back edge 258, a left side edge 257a, and a laterally opposing right side edge 257b. Any or all of the edges of the absorbent core 250 may lie inward of, or may coincide with, the respective edges of the absorbent assembly 200. For example, in the exemplary absorbent assembly 200 shown in FIG. 40, the side edges 257 of the absorbent core 250 are located laterally inward of the side edges 237 of the absorbent assembly 200, while the front edge 256 and back edge 258 of the absorbent core 250 coincide with the respective front edge 236 and back edge 238 of the absorbent assembly 200.

The absorbent assembly 200 may be attached to the chassis 100 over any part or the whole of the area of the absorbent assembly 200. The absorbent assembly 200 may be attached on its exterior surface 204 to the chassis 100 in a cruciform attachment pattern, i.e., in an attachment pattern that forms or is arranged in a cross or “+” shape. The cruciform attachment pattern may be contiguous, i.e., all of its portions may be touching or connected throughout the pattern in an unbroken sequence. Alternatively, the cruciform attachment pattern may include detached portions and thereby lack contiguity but still be arranged such that the shape of the overall pattern is a cruciform. For example, a discontiguous cruciform attachment pattern may include a longitudinally extending portion disposed along the longitudinal axis and separate left and right laterally distal portions disposed along or adjacent to the lateral axis and thereby form a cruciform as the shape of the overall pattern.

An exemplary contiguous cruciform attachment pattern 210 is shown in FIG. 40. The portions 190 of the chassis 100 that lie outside such a cruciform attachment pattern are not restrained by attachment to the absorbent assembly 200 and therefore remain extensible. In particular, a relatively narrow longitudinally extending portion 212 of a cruciform attachment pattern 210 like that shown in FIG. 40 leaves the majority of the width of the chassis 100 in the front waist region 36 and in the back waist region 38 freely extensible and thereby allows extension of the chassis 100 in the lateral direction in these regions. A relatively wide laterally extending portion 214 of a cruciform attachment pattern 210 prevents the portion of the chassis 100 in the crotch region 37 to which the absorbent assembly 200 may be attached from shifting relative to the absorbent assembly 200 in that region. A relatively wide laterally extending portion 214 of a cruciform attachment pattern 210 may also contribute to the effectiveness of the side flaps 147 when the elastic strands 167 lift the proximal edges 157 into contact with the body of the wearer. Because the relatively wide laterally extending portion 214 of the cruciform attachment pattern 210 restrains the chassis 100 over a relatively wide portion of the width of the crotch region 37, the side flaps 147 are better supported at their bases while being lifted by the elastic strands 167

Within the extent of the cruciform attachment pattern 210, the absorbent assembly 200 may be attached to the chassis 100 continuously or intermittently. For example, a film of an adhesive material may be applied continuously over the entire area of the cruciform attachment pattern and then used to continuously attach the absorbent assembly to the chassis. As an alternative example, an adhesive material may be applied discontinuously at and inside the boundaries of the cruciform attachment pattern, such as in the form of dots, stripes, beads, spirals, etc., and then used to attach the absorbent assembly to the chassis.

Cruciform attachment pattern 210 may be disposed either symmetrically or asymmetrically with respect to either or both of the longitudinal axis 42 and the lateral axis 44 of the chassis 100. In addition, the cruciform attachment pattern 210 may be disposed symmetrically or asymmetrically with respect to either or both of the side edges 237 and the front edge 236 and the back edge 238 of the absorbent assembly 200.

Suitable configurations of cruciform attachment patterns are disclosed in U.S. Pat. No. 6,962,578 to LaVon issued on 8 Nov. 2005.

Alternatively, the absorbent assembly 200 may be attached to the interior surface 102 of the chassis 100 in a convexly-shaped attachment pattern 210′, which may be in the shape of an oval or may be egg-shaped. The convexly-shaped attachment pattern 210′ may be contiguous, i.e., all of its portions may be touching or connected throughout the pattern in an unbroken sequence.

Alternatively, it may include detached portions and thereby lack contiguity but still be arranged such that the shape of the overall pattern is in the form of, for example, an oval. For example, a discontiguous convexly-shaped attachment pattern 210′ may include a longitudinally extending portion disposed along the longitudinal axis 42 and separate left and right laterally spaced portions disposed along or adjacent to the lateral axis 44, the laterally spaced portions having extending longitudinally to different lengths thereby forming an oval shaped pattern. In one embodiment, shown in FIG. 64, the oval-like attachment pattern 210 comprises 5 laterally spaced stripes of adhesive 624-628 attaching the absorbent assembly 200 to the interior surface 102 of the chassis 100. The central stripe 626 may be disposed at or adjacent the longitudinal axis 42 and is shown as having the greatest longitudinal extent. The most distal of the adhesive stripes 624 and 628 are shown as having the least longitudinal extent and the intermediate stripes 625 and 627, those located between the distal stripes 624 and 628 and the central stripe 626 are shown having a longitudinal extent between that of the central stripe 626 and the distal stripes 624 and 628.

The portions of the chassis 100 that lie outside such a convexly-shaped attachment pattern 210 or oval-like attachment pattern 210 are not restrained by attachment to the absorbent assembly 200 and therefore remain extensible. Thus, in embodiments where the absorbent assembly 200 is attached to the backsheet 26 in the convexly-shaped attachment pattern 210, the backsheet 26 substantially loses its extensibility in the area of attachment. The portion of the backsheet 26 disposed outside of the convexly-shaped attachment pattern 210 may remain extensible. Thus, it may be desirable to activate the backsheet 26 in a pattern complimentary with a given attachment pattern (e.g., a concave activation pattern 675 to compliment a convexly-shaped or oval-like attachment patterns 210).

Within the extent of the shaped attachment patterns (e.g., the cruciform, convexly-shaped, and oval-like attachment patterns 210), the absorbent assembly 200 may be attached to the chassis 100 continuously or intermittently as shown in FIGS. 64 and 65. For example, a film of adhesive (not shown) may be applied continuously over the entire area of the cruciform attachment pattern 210 and then used to continuously attach the absorbent assembly 200 to the chassis 100. As an alternative example, an adhesive may be applied in a laterally and/or longitudinally discontinuous pattern at and inside the boundaries of the convexly shaped attachment pattern 210″, such as in the form of dots, stripes (e.g., stripes 624-628), beads, spirals, etc., and then used to attach the absorbent assembly 200 to the chassis 100. Laterally and/or longitudinally discontinuous patterns may allow fluid to flow and/or be temporarily retained under the absorbent assembly until absorbed through the lower covering sheet.

The shaped attachment patterns (e.g., the cruciform, convexly-shaped, and oval-like attachment patterns 210) may be disposed symmetrically or asymmetrically with respect to either or both of the longitudinal axis 42 and the lateral axis 44 of the chassis 100. In addition, the shaped attachment patterns (e.g., the cruciform, convexly-shaped, and oval-like attachment patterns 210) may be disposed symmetrically or asymmetrically with respect to either or both of the side edges 237a and b and the front and back edges 236 and 238 of the absorbent assembly 200. Other suitable configurations of cruciform attachment patterns 210 are disclosed in U.S. Pat. No. 6,962,578 issued on 8 Nov. 2005.

The absorbent core 250 may be disposed between a lower covering sheet that may be disposed on the exterior face of the absorbent core 250 in a face-to-face arrangement with the interior surface 102 of the chassis and an upper covering sheet that may be disposed on the interior face of the absorbent core 250. Such an upper covering sheet and lower covering sheet may be attached together to contain the absorbent core 250 between them and thereby form the absorbent assembly 200. For example, in the exemplary absorbent assembly 200 shown in the figures, an upper covering sheet 24 and a lower covering sheet 25 are attached together at or adjacent to the side edges 237 of the absorbent assembly 200 in adhesive attachment zones 29. Alternatively, the upper covering sheet 24 and the lower covering sheet 25 may be attached together in places other than the side edges 237 of the absorbent assembly 200, e.g., at or adjacent to the end edges 236 and 238, or at or adjacent to both the end edges 236 and 238 and the side edges 237.

The upper covering sheet 24 may be water-permeable and allows liquid waste to pass through to the absorbent core 250, where the liquid waste may be absorbed. The lower covering sheet 25 may be water-impermeable. However, the lower covering sheet 25 may be water-permeable.

In the exemplary absorbent assembly 200 shown in FIG. 41, FIG. 42, and FIG. 43, the upper covering sheet 24 and the lower covering sheet 25 are of the same size, i.e., both the upper covering sheet 24 and the lower covering sheet 25 extend to the front edge 236 and back edge 238, as well as to the side edges 237 of the absorbent assembly 200. Alternatively, the upper covering sheet 24 and the lower covering sheet 25 may differ in size.

As another example, the upper covering sheet 24 may be larger than the lower covering sheet 25 and may be wrapped over the side edges 257 of the absorbent core 250 onto the interior surface of the absorbent core 250, where the upper covering sheet 24 and the lower covering sheet 25 may be attached together. Alternatively, in place of a separate upper covering sheet 24 and a separate lower covering sheet 25, a single covering sheet may be wrapped around the absorbent core 250 and attached to itself to contain the absorbent core 250.

The upper covering sheet 24 may also comprise graphics. The graphics on the upper covering sheet 24 may be random and form a longitudinally extending continuously repeating pattern (not shown). Alternatively, the graphics may be designed for a specific size absorbent article or to provide specific design language associated with a specific region of the absorbent article. Such graphics are often referred to as pitched graphics, meaning the design is linked or associated with the pitch (i.e., length of the absorbent article) and may provides a visual signal for the user allowing them to identify and ensure proper application and initial position of the absorbent assembly 200 on the wearer.

The upper covering sheet 24 may also comprise a lotion. The lotion may be in a pattern. The pattern may be laterally and/or longitudinally discontinuous. The lotion pattern may extend from the front waist edge 136 to the back waist edge 138. Alternatively, the lotion pattern may extend from the front edge of the absorbent assembly 236 to the back edge 238 of the absorbent assembly 200. Examples of suitable lotions include, but are not limited to, those described in U.S. Pat. Nos. 5,607,760 to Roe on; U.S. Pat. No. 5,609,587 to Roe; U.S. Pat. No. 5,635,191 to Roe et al.; U.S. Pat. No. 5,643,588 to Roe et al.; and U.S. Pat. No. 5,968,025 to Roe et al. The lotion may also be incorporated onto the cuff flaps.

The absorbent core may include one or more acquisition components in addition to one or more storage components. The absorbent core acquisition component serves to acquire deposited liquid bodily waste material and transfer it to the absorbent core storage component. Any porous absorbent material which will imbibe and partition liquid bodily waste material to the storage component or components may be used to form the acquisition component. Preferred materials for the acquisition component include synthetic fiber materials, open celled polymeric foam materials, fibrous nonwoven materials, cellulosic nonwoven materials, and various combination synthetic/cellulosic nonwoven materials. Examples of such acquisition materials are more fully described in U.S. Pat. No. 4,950,264 issued on Aug. 21, 1990.

Such an acquisition component 290 overlying an absorbent core storage component 272 is shown in FIG. 43. A separation sheet 292 of, e.g., a tissue or a nonwoven material, may be disposed between the absorbent core storage component 272 and the absorbent core acquisition component 290 to help ensure that none of the gel formed by a superabsorbent polymer that may be included in the absorbent core storage component reaches the skin of the wearer.

Suitable well-known absorbent materials for the absorbent core include cellulose fibers in the form of comminuted wood pulp, which is commonly known as “airfelt”, layers or sheets of natural or synthetic fibrous material, superabsorbent polymer, etc. These absorbent materials may be used separately or in combination and many may be used in a discrete form, i.e., in the form of fibers, granules, particles, layers and the like.

The discrete form of an absorbent material may be immobilized in pockets formed by a layer of a thermoplastic material, such as a hot melt adhesive, that intermittently contacts and adheres to a substrate, such as a covering sheet, while diverging away from the substrate at the pockets. Absorbent core components having such structures and being suitable for the storage of liquid bodily wastes are described in U.S. Patent Application Publication Nos. 2004/0162536 dated 19 Aug. 2004 and 2004/0167486 dated 26 Aug. 2004, as well as U.S. Application Nos. 60/936,102, to Hundorf, dated Jun. 18, 2007, 60/936,109, to Hundorf, dated Jun. 18, 2007, 60/936,149, to Hundorf, dated Jun. 18, 2007, 60/936,036, to Hundorf, dated Jun. 18, 2007, 60/936,068, to Hundorf, dated Jun. 18, 2007, 60/936,150, to Hundorf, dated Jun. 18, 2007, 60/936,085, to Ashton, dated Jun. 18, 2007, 60/936,084, to Ashton, dated Jun. 18, 2007, 60/936,146, to Ashton, dated Jun. 18, 2007, and 61/091,799 to Hundorf filed on Aug. 26, 2008.

An exemplary absorbent assembly 200 having such a structure is shown in FIG. 44. In this absorbent assembly 200, the absorbent core 250 includes particles of superabsorbent polymer 270 that are contained inside pockets 280 formed by a layer 275 of a thermoplastic material. The layer 275 of the thermoplastic material intermittently contacts and adheres to a substrate sheet 274 at the areas of attachment 282. Between the areas of attachment 282, the layer 275 diverges away from the substrate sheet 274 to form the pockets 280. The layer 275 may have the form of a sheet of fibers of the thermoplastic material through which the liquid waste may pass to the particles of superabsorbent polymer 270 to be absorbed.

In FIG. 44, a separate thermoplastic layer covering sheet 276 is shown overlying the layer 275 of the thermoplastic material. Alternatively, the separate thermoplastic layer covering sheet may be omitted.

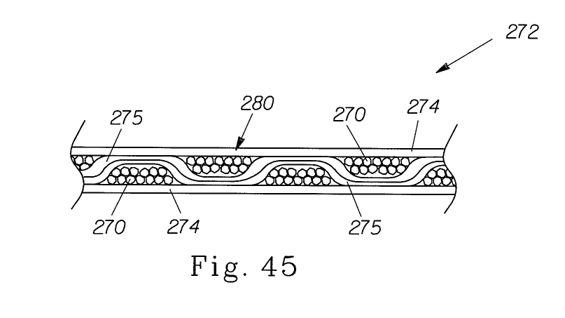

As another alternative, as shown in FIG. 45, two absorbent core storage components 272 may be superposed with one absorbent core storage component 272 inverted such that its pockets 280 nest into the recesses at the areas of attachment 282 of the other absorbent core storage component 272 and the respective single substrate sheets 274 distally oppose each other. In such a combined absorbent assembly 200, the distally opposing single substrate sheets 274 may serve respectively as the upper covering sheet 24 and the lower covering sheet 25.

Alternatively, as disclosed in U.S. Ser. No. 61/091,799 filed on Aug. 26, 2008, the absorbent assembly may comprise superabsorbent polymer material deposited from a plurality of reservoirs in a printing roll onto a substrate disposed on a grid of a support which includes a plurality of cross bars extending substantially parallel to and spaced from one another so as to form channels extending between the plurality of cross bars. The superabsorbent polymer material may be arranged in an array comprising rows extending substantially parallel to and spaced from one another. A thermoplastic adhesive material may be deposited on the superabsorbent polymer material and the substrate to cover the superabsorbent polymer material on the substrate and form an absorbent layer.

Further, a wetness indicator composition, such as disclosed in U.S. Ser. No. 61/168,756 filed on Apr. 13, 2009, may be incorporated in the absorbent assembly, as well as other components of the absorbent article. The wetness indicator composition may comprise a stabilizer, a colorant, and a matrix. The absorbent core may comprise a nonwoven layer and a complex of absorbent polymer material and thermoplastic adhesive material. The wetness indicator composition may be in direct contact with an inner surface of the backsheet and an outer surface of the nonwoven layer. And, the complex of absorbent polymer material and thermoplastic adhesive material may be in direct contact with an inner surface of the nonwoven. Further, the absorbent core may be cellulose free.

Additionally, the absorbent article, and particularly including the absorbent assembly, may comprise an odor control system as disclosed in U.S. Ser. Nos. 12/272,967 filed on Nov. 18, 2008 and 61/116,490 filed on Nov. 20, 2008. The odor control system may comprise a bleach activator system. The bleach activator system may comprise a peroxygen bleaching compound and a bleach activator capable of reacting with the peroxygen bleaching compound to form a peracid. The peroxygen bleaching compound may be a source of hydrogen peroxide.

### STATEMENTS OF INCORPORATION BY REFERENCE AND INTENDED SCOPE OF CLAIMS

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a front waist region, a back waist region, a crotch region between the waist regions, a front waist end edge, and a back waist end edge;
a longitudinal axis extending from a midpoint of the front waist end edge through the crotch region to a midpoint of the longitudinally opposed back waist end edge;
a web comprising an interior surface and an exterior surface;
an absorbent assembly comprising an interior surface and an exterior surface;
wherein the web is folded over at a first fold line forming a first hem;
wherein the web is folded over at a second fold line forming a second hem;
wherein the web is folded over at a third fold line and attached in the front and back waist regions forming a first side flap, the third fold line forming a first side edge of the absorbent article;
wherein the web is folded over at a fourth fold line and attached in the front and back waist regions forming a second side flap, the fourth fold line forming a second side edge of the absorbent article;
wherein the first side flap comprises the first hem and wherein the second side flap comprises the second hem;
wherein each of the first and second hems comprise a longitudinally extending gathering member;
wherein the first fold line forms a proximal edge of the first side flap and the second fold line forms a proximal edge of the second side flap;
wherein the first and second proximal edges are disposed laterally inward of the first and second side edges of the absorbent article;
wherein the first and second proximal edges of the first and second side flaps are disposed between the longitudinal centerline and the first and second side edges of the absorbent article, respectively;
the absorbent article also comprising a second elastic gathering member attached at or adjacent the side edge of the absorbent article; and
the absorbent article also comprising laterally opposing deployable belt ears attached to the web in at least one of the waist regions, each belt ear being disposed laterally inwardly until being deployed laterally outward so as to project laterally outward beyond the respective distal edges of the side flaps.

2. The disposable diaper of claim 1 wherein the chassis comprises longitudinally opposing belt ears.

3. The disposable diaper of claim 1 wherein the chassis includes fastening elements disposed on at least two of the belt ears and adapted for fastening the front waist region to the back waist region to encircle a waist and legs of a wearer.

4. The disposable diaper of claim 3 wherein the fastening elements comprise mechanical fastener.

5. The disposable diaper of claim 1 wherein the web comprises a first layer and a second layer.

6. The disposable diaper of claim 5, wherein at least one of the layers is folded to form the side flaps.

7. The disposable diaper of claim 5 wherein the first layer is a nonwoven and the second layer is a film and wherein at least a portion of the side flap is formed by one or both of the first and second layers.

8. The disposable diaper of claim 4, wherein the mechanical fastener is adapted to engage with the backsheet nonwoven.

9. The disposable diaper of claim 1 wherein the extensible portion of the chassis comprises a formed web material including at least two distinct laterally extending embossed regions each containing a pattern of generally longitudinally oriented alternating ridges and valleys created by an embossment and also containing an unembossed region located between the embossed regions, such that the extensible portion of the chassis can be laterally extended to a given extent with the application of relatively less force than that required to laterally extend the same portion of the chassis to the same given extent before the embossment.

10. The disposable diaper of claim 1 wherein the chassis comprises a first activation pattern disposed in at least one of the front waist region, back waist region, and crotch region; and wherein the chassis comprises a second activation pattern disposed in the front waist region and/or the back waist region.

11. A disposable absorbent article comprising:
an absorbent assembly comprising an absorbent core;
a chassis comprising a longitudinal axis, a lateral axis, a front waist region comprising a front waist edge, a back waist region comprising a back waist edge, a crotch region between the waist regions, laterally opposing side edges extending between the front waist edge and the back waist edge, the side edges comprising folded side edge segments disposed in the front and back waist regions and cut side edge segments disposed in the crotch region, an exterior surface, and an interior surface to which the absorbent assembly is attached, the chassis comprising a water-impermeable backsheet and laterally opposing side flaps each of the side flaps comprising a longitudinally extending first elastic gathering member attached at or adjacent to its proximal edge;
the chassis further comprising a barrier attachment zone disposed between a side edge of the absorbent assembly and the side edge of the chassis, the barrier attachment zone extending continuously from the front waist region through the crotch region to the back waist region and forming an area of attachment between the side flaps and the backsheet;
the chassis also comprising laterally opposing deployable belt ears attached to the chassis in at least one of the waist regions, each belt ear being disposed laterally inwardly until being deployed laterally outward so as to project laterally outward beyond the respective distal edges of the barrier attachment zone; and
the chassis also comprising a second elastic gathering member disposed between the side barrier attachment zone and the side edge of the chassis.
